(19) United States
(12) Patent Application Publication
(10) Pub. No.: US 2023/0270547 A1
(43) Pub. Date: Aug. 31, 2023

(54) EXPANDABLE SHEATH INCLUDING SMART INTRODUCER

(71) Applicant: EDWARDS LIFESCIENCES CORPORATION, Irvine, CA (US)

(72) Inventor: Ilan Tamir, Irvine, CA (US)

(21) Appl. No.: 18/133,723

(22) Filed: Apr. 12, 2023

Related U.S. Application Data
(63) Continuation of application No. PCT/US2021/050012, filed on Sep. 13, 2021.
(60) Provisional application No. 63/091,226, filed on Oct. 13, 2020.

(51) Int. Cl. A61F 2/24

(52) U.S. Cl. CPC A61F 2/2436; A61M 2025/0042

(57) ABSTRACT

An introducer sheath system is disclosed herein that includes an introducer locking hub, an introducer coupled to the introducer locking hub, and a hypotube disposed within the central lumen of the introducer. The introducer locking hub prevents axial and rotational translation of the introducer with respect to the sheath. The introducer defines an elongated body having a proximal piece and a separate distal piece, the proximal piece coupled to the introducer locking hub and the distal piece coupled to the distal end of the hypotube. The body portion of the hypotube is slidably disposed within the central lumen of the proximal piece of the introducer such that movement of the hypotube within the central lumen of the proximal piece causes the distal piece of the introducer to move with respect to the proximal piece.

### CROSS-REFERENCE TO RELATED APPLICATIONS

This application is a continuation of International Application No. PCT/US2021/050012, filed Sep. 13, 2021, which claims the benefit of U.S. Provisional Application No. 63/091,226, filed Oct. 13, 2020, the contents of which are incorporated herein by reference in their entirety.

### FIELD

The present application is directed to a sheath for use with catheter-based technologies for repairing and/or replacing heart valves, as well as for delivering an implant, such as a prosthetic valve to a heart via the patient’s vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques. Percutaneous interventional medical procedures utilize the large blood vessels of the body reach target destinations rather than surgically opening target site. There are many types of diseases states that can be treated via interventional methods including coronary blockages, valve replacements (TAVR) and brain aneurysms. These techniques involve using wires, catheters, balloons, electrodes and other thin devices to travel down the length of the blood vessels from the access site to the target site. The devices have a proximal end which the clinician controls outside of the body and a distal end inside the body which is responsible for treating the disease state. Percutaneous interventional procedures offer several advantages over open surgical techniques. First, they require smaller incision sites which reduces scarring and bleeding as well as infection risk. Procedures are also less traumatic to the tissue, so recovery times are reduced. Finally, interventional techniques can usually be performed much faster, and with fewer clinicians participating in the procedure, so overall costs are lowered. In some cases, the need for anesthesia is also eliminated, further speeding up the recovery process and reducing risk.

A single procedure typically uses several different guidewires, catheters, and balloons to achieve the desired effect. One at a time, each tool is inserted and then removed from the access site sequentially. For example, a guidewire is used to track to the correct location within the body. Next a balloon may be used to dilate a section of narrowed blood vessel. Last, an implant may be delivered to the target site. Because catheters are frequently inserted and removed, introducer sheaths are used to protect the local anatomy and simplify the procedure.

An introducer sheath can be used to safely introduce a delivery apparatus into a patient’s vasculature (e.g., the femoral artery). Introducer sheaths are conduits that seal onto the access site blood vessel to reduce bleeding and trauma to the vessel caused by catheters with rough edges. An introducer sheath generally has an elongated sleeve that is inserted into the vasculature and a housing that contains one or more sealing valves that allow a delivery apparatus to be placed in fluid communication with the vasculature with minimal blood loss. Once the introducer sheath is positioned within the vasculature, the shaft of the delivery apparatus is advanced through the sheath and into the vasculature, carrying the prosthetic device. Expandable introducer sheaths, formed of highly elastomeric materials, allow for the dilating of the vessel to be performed by the passing prosthetic device. Expandable introducer sheaths are disclosed in U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. Application No. 14/880,109, entitled “Expandable Sheath,” U.S. Application No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. Application No. 15/997,587, entitled “Expandable Sheath for Introducing an Endovascular Delivery Device into a Body,” U.S. Application No. 16/378,417, entitled “Expandable Sheath,” the disclosures of which are herein incorporated by reference.

Conventional methods of accessing a vessel, such as a femoral artery, prior to introducing the delivery system include dilating the vessel using multiple dilators or sheaths that progressively increase in diameter. Typically, the introducer is inserted into the sheath during preparation and both are then inserted into the vessel. Due to the need for a smooth transition from the introducer to the sheath, it is vital that the change in diameter of the introducer occurs distal to the tip of the sheath, such that the tip of the sheath fits snugly around the diameter. During insertion of the sheath and introducer, it is possible that the introducer can move backwards within the sheath, eliminating the aforementioned snug fit and creating a lip between the sheath tip and the smaller outer diameter of the introducer. This lip/gap can lead to severe vessel trauma during insertion.

Also, many introducer sheaths are optimized for the pre insertion phase instead of the post insertion phase of the delivery. These introducer sheaths are not suited for retrieval and/or repositioning of medical devices (e.g., heart valve, balloon) or other components of a delivery system. The distal tip of the sheath may contract or catch on the passing medical equipment, causing trauma to the patient tissue or damage to the sheath or medical device.

Moreover, some procedures, such as a transseptal approach for mitral valve replacement/repair, require prolonged dilation of incisions in heart tissue and a curving/bending of the sheath to access the treatment site, prolonging procedure time and recovering and increasing risk of trauma to vessels and heart tissue.

Accordingly, there remains a need for further improvements in expandable introducer sheath for endovascular systems used to implant valves and other prosthetic devices.

Furthermore, there also remains a need for further improvements to introducers and expandable introducer sheaths that are optimized for the post insertion phase of delivery of valves and other prosthetic devices into endovascular systems.

### SUMMARY

The introducer sheath system disclosed herein comprises an introducer locking hub comprising a hub body having a proximal end and a distal end and defining a central lumen extending longitudinally between the proximal end and the distal end; an introducer coupled to the introducer locking hub, the introducer extending beyond the distal end of the hub body and having a central lumen extending therethrough, the introducer defining an elongated body comprising a proximal piece and a separate distal piece, the proximal piece coupled to the introducer locking hub; and a hypotube disposed within the central lumen of the introducer, the hypotube having a proximal end and a distal end and defining a central lumen extending longitudinally between the proximal end and the distal end, wherein the distal end of the hypotube is coupled to the distal piece of the introducer and at least a portion of the hypotube is slidably disposed in the central lumen of the proximal piece of the introducer such that movement of the hypotube within the central lumen of the proximal piece causes the distal piece of the introducer to move with respect to the proximal piece of the introducer.

A method of delivering a prosthetic device to a procedure site is disclosed herein, the method includes: providing an introducer locking hub having an elongated introducer coupled to a hub body of the locking hub, the introducer comprising a proximal piece and a separate distal piece, the proximal piece being coupled to the hub body of the locking hub, the separate distal piece being coupled to a hypotube, the hypotube slidably disposed within a central lumen of the proximal piece; advancing the introducer through the central lumen of an expandable sheath, the expandable sheath defining an elongated tubular body coupled to a sheath hub and a distal tip portion provided at a distal end of the elongated tubular body; coupling the introducer locking hub to a sheath hub such that the axial position of the introducer locking hub and the sheath hub is fixed; inserting the coupled sheath and introducer at least partially into the vasculature of the patient; advancing the hypotube within the central lumen of the proximal piece of the introducer such that the distal piece moves from a first position, adjacent the proximal piece, to a second position where the distal piece is spaced from the proximal piece; expanding the distal tip portion of the expandable sheath; withdrawing the hypotube within the central lumen of the proximal piece of the introducer such that the distal piece of the introducer abuts the proximal piece of the introducer; withdrawing the introducer from the central lumen of the sheath; advancing a medical device through the central lumen of the sheath; and delivering the medical device to a procedure site via the central lumen of the sheath.

A method of expanding the distal tip of an expandable sheath is disclosed herein, the method includes: providing an introducer locking hub having an elongated introducer coupled to a hub body of the locking hub, the introducer comprising a proximal piece and a separate distal piece, the proximal piece being coupled to the hub body of the locking hub, the separate distal piece being coupled to a hypotube, the hypotube slidably disposed within a central lumen of the hub body; advancing the introducer through the central lumen of an expandable sheath, the expandable sheath defining an elongated tubular body coupled to a sheath hub and a distal tip portion provided at a distal end of the elongated tubular body; coupling the introducer locking hub to a sheath hub such that the axial position of the introducer locking hub and the sheath hub is fixed; advancing the hypotube within the central lumen of the proximal piece of the introducer such that the distal piece moves from a first position, adjacent the proximal piece, to a second position where the distal piece is spaced from the proximal piece; and expanding the distal tip portion of the expandable sheath.

### DESCRIPTION OF DRAWINGS

FIG. 1 is an elevation view of an expandable sheath along with an endovascular delivery apparatus for implanting a prosthetic implant.

FIG. 2 is an elevation view of an expandable sheath including an introducer locking hub, a sheath locking sleeve, and an introducer.

FIG. 3 is an elevation view of the expandable sheath of FIG. 2 along with an endovascular delivery apparatus for implanting a prosthetic implant.

FIG. 4 is an elevation view of an expandable sheath a sheath hub, an introducer locking hub, and a sheath locking sleeve of FIG. 2.

FIG. 5A is a cross sectional view of the sheath hub, introducer locking hub, and sheath locking sleeve of FIG. 2.

FIG. 5B is a cross sectional view of the introducer cap, the sheath hub, the introducer locking hub, the sheath locking sleeve of FIG. 2.

FIG. 6 is a cross sectional view of the introducer cap, sheath hub, introducer locking hub, and sheath locking sleeve of FIG. 2.

FIG. 7 is a distal end view of the sheath locking sleeve of FIG. 2 and the proximal fluid seal of FIGS. 5A-5B.

FIG. 8A is a first elevation view of the introducer locking hub of FIG. 2 coupled to an introducer.

FIG. 8B is a second elevation view of the introducer locking hub of FIG. 2 coupled to the introducer.

FIG. 8C is a distal end view of the introducer locking hub of FIG. 2 coupled to the introducer.

FIG. 8D is a partial side view of the introducer locking hub of FIG. 2 coupled to the introducer.

FIG. 8E is a partial perspective view of the introducer locking hub of FIG. 2 coupled to the introducer.

FIG. 8F is a partial perspective view of the introducer locking hub of FIG. 2 coupled to the introducer.

FIG. 9A is a distal end view of the introducer locking hub of FIG. 2.

FIG. 9B is a first elevation view of the introducer locking hub of FIG. 2.

FIG. 9C is a proximal end view of the introducer locking hub of FIG. 2.

FIG. 9D is a first perspective view of the introducer locking hub of FIG. 2.

FIG. 9E is a second elevation view of the introducer locking hub of FIG. 2.

FIG. 9F is a second perspective view of the introducer locking hub of FIG. 2.

FIG. 10A is a distal end view of the sheath locking sleeve of FIG. 2.

FIG. 10B is a first elevation view of the sheath locking sleeve of FIG. 2.

FIG. 10C is a proximal end view of the sheath locking sleeve of FIG. 2.

FIG. 10D is a first perspective view of the sheath locking sleeve of FIG. 2.

FIG. 10E is a second elevation view of the sheath locking sleeve of FIG. 2.

FIG. 10F is a second perspective view of the sheath locking sleeve of FIG. 2.

FIG. 11 is a side elevation cross-sectional view of a portion of the expandable sheath of FIG. 3.

FIG. 12 is a magnified view of a portion of the expandable sheath of FIG. 3.

FIG. 13A is a magnified view of a portion of the expandable sheath of FIG. 3 with the outer layer removed for purposes of illustration.

FIG. 13B is a magnified view of a portion of the braided layer of the sheath of FIG. 3.

FIG. 14 is a magnified view of a portion of the expandable sheath of FIG. 3 illustrating expansion of the sheath as a prosthetic device is advanced through the sheath.

FIGS. 15A-15C are side views of the expandable sheath of FIG. 3 including a delivery device and implant.

FIG. 16 is a schematic representation of an introducer locking hub, a hypotube, and an introducer.

FIG. 17 is a schematic representation of a sheath hub, sheath locking sleeve and an expandable sheath with a pre-expanded distal end.

FIG. 18A is a schematic representation of the introducer locking hub, hypotube, and introducer of FIG. 16 coupled to the sheath hub, sheath locking sleeve, and expandable sheath of FIG. 17 in a first position.

FIG. 18B is a schematic representation of the example system of FIG. 18A in a second position.

FIG. 19 is an elevation view of an introducer locking hub, a hypotube, and an introducer.

FIG. 20 is an elevation view of a sheath hub, sheath locking sleeve, and an expandable sheath.

FIG. 21A is an elevation view of the sheath hub, sheath locking sleeve, and expandable sheath of FIG. 20 removably coupled to the introducer locking hub, hypotube, and introducer of FIG. 19 in a first position.

FIG. 21B is a magnified view of the distal end of the example system of FIG. 21A in the first position.

FIG. 21C is a magnified view of the distal end of the example system of FIG. 21A in the first position with a transparent distal piece of the introducer.

FIG. 21D is a partial cross section view of the introducer locking hub, hypotube, and introducer of FIG. 21A in the first position.

FIG. 22A is an elevation view of the sheath hub, sheath locking sleeve, and expandable sheath of FIG. 20 removably coupled to the introducer locking hub, hypotube, and introducer of FIG. 19 in a second position.

FIG. 22B is a magnified view of the distal end of the example system of FIG. 22A in the second position.

FIG. 22C is a partial cross section view of the introducer locking hub, hypotube, and introducer of FIG. 22A in the second position.

FIG. 23A is a side elevation view of the sheath hub, sheath locking sleeve, and expandable sheath of FIG. 20 removably coupled to the introducer locking hub, hypotube, and introducer of FIG. 19 in a first position.

FIG. 23B is a side elevation view of the sheath hub, sheath locking sleeve, and expandable sheath of FIG. 20 removably coupled to the introducer locking hub, hypotube, and introducer of FIG. 19 in a second position.

FIG. 24A is a magnified view of a hypotube and a transparent introducer locking hub in a first position.

FIG. 24B is a magnified view of a hypotube and a transparent introducer locking hub in a second position.

FIG. 25A is a distal end view of the slider of FIG. 19.

FIG. 25B is a first elevation view of the slider of FIG. 19.

FIG. 25C is a proximal end view of the slider of FIG. 19.

FIG. 25D is a first perspective view of the slider of FIG. 19.

FIG. 25E is a second elevation view of the slider of FIG. 19.

FIG. 25F is a second perspective view of the slider of FIG. 19.

FIG. 26A is a first perspective view of the introducer locking hub of FIG. 19.

FIG. 26B is a second perspective view of the introducer locking hub of FIG. 19.

FIG. 27A is a magnified view of an introducer locking hub, a sheath hub, and a safety lock in a first position.

FIG. 27B is a magnified view of a distal end of the example system of FIG. 27A.

FIG. 28A is a magnified view of the introducer locking hub, sheath hub, and safety lock of FIG. 27A in a second position.

FIG. 28B is a magnified view of the corresponding distal end of FIG. 28A.

FIG. 29 is a magnified view of the removal of the safety lock from the example system of FIG. 27A.

FIG. 30A is a magnified view of the introducer locking hub, sheath hub, and safety lock of FIG. 27A in the first position.

FIG. 30B is a magnified view of the corresponding distal end of FIG. 30A in the first position but no longer retaining a distal end of the sheath.

FIG. 31 is a magnified view of the combination of FIG. 27A with the safety lock removed and the introducer locking hub unlocked from the sheath hub.

FIG. 32 is an elevation view of a sheath locking sleeve of the sheath hub.

FIG. 33 is a section view of an introducer locking hub.

FIG. 34 is a magnified view of a slider, introducer, and spring.

FIG. 35A is a magnified view of a transparent introducer locking hub, sheath locking spring and hypotube in a first position.

FIG. 35B is a magnified view of a distal end of the example system of FIG. 35A in the first position

FIG. 36A is a magnified view of the example system of FIG. 35A in a second position.

FIG. 36B is a cross section view of the example system of 35A in the second position.

FIG. 36C is a magnified view of a distal end of the example system of FIG. 35A in the second position.

### DETAILED DESCRIPTION

The following description of certain examples of the inventive concepts should not be used to limit the scope of the claims. Other examples, features, aspects, examples, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the examples of this disclosure are described herein. The described methods, systems, and apparatus should not be construed as limiting in any way. Instead, the present disclosure is directed toward all novel and nonobvious features and aspects of the various disclosed examples, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect, example or example are to be understood to be applicable to any other aspect, or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The aspects herein are not restricted to the details of any foregoing examples. The aspects herein extend to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

Disclosed examples of an expandable sheath can minimize trauma to the vessel by allowing for temporary expansion of a portion of the introducer sheath to accommodate the delivery system, followed by a return to the original diameter once the device passes through. Disclosed examples of the introducer sheath prevent the introducer from separating from the sheath during insertion by locking of the proximal hub of the introducer to the proximal hub of the sheath. Fixing the introducer and the sheath prevents the introducer from moving backward during insertion, thereby maintaining a snug fit and smooth transition between the introducer and the distal end of the sheath. Furthermore, present examples can reduce the length of time a procedure takes, as well as reduce the risk of a longitudinal or radial vessel tear, or plaque dislodgement because only one sheath is required, rather than several different sizes of sheaths. Examples of the present expandable sheath can avoid the need for multiple insertions for the dilation of the vessel.

Disclosed herein are elongate introducer sheaths that are particularly suitable for delivery of implants in the form of implantable heart valves, such as balloon-expandable implantable heart valves. Balloon-expandable implantable heart valves are well-known and will not be described in detail here. An example of such an implantable heart valve is described in U.S. Pat. No. 5,411,552, and also in U.S. Pat. No. 9,393,110, both of which are hereby incorporated by reference. The expandable introducer sheaths disclosed herein may also be used to deliver other types of implantable medical device, such as self-expanding and mechanically expanding implantable heart valves, stents or filters. Beyond transcatheter heart valves, the introducer sheath system can be useful for other types of minimally invasive surgery, such as any surgery requiring introduction of an apparatus into a subject’s vessel. For example, the introducer sheath system can be used to introduce other types of delivery apparatus for placing various types of intraluminal devices (e.g., stents, stented grafts, balloon catheters for angioplasty procedures, etc.) into many types of vascular and non-vascular body lumens (e.g., veins, arteries, esophagus, ducts of the biliary tree, intestine, urethra, fallopian tube, other endocrine or exocrine ducts, etc.). The term “implantable” as used herein is broadly defined to mean anything – prosthetic or not – that is delivered to a site within a body. A diagnostic device, for example, may be an implantable.

Disclosed herein are pre-expanded introducer sheaths that are optimized for post insertion and delivery of implants. For example, the pre-expanded introducer sheaths can minimize balloon retrieval force, support burst balloon retrieval, and support bailout procedure (e.g. crimped valve retrieval). Also disclosed herein are introducers for use with the pre-expanded introducer sheaths such the transition between the introducers and the pre-expanded introducer sheaths is as smooth as possible.

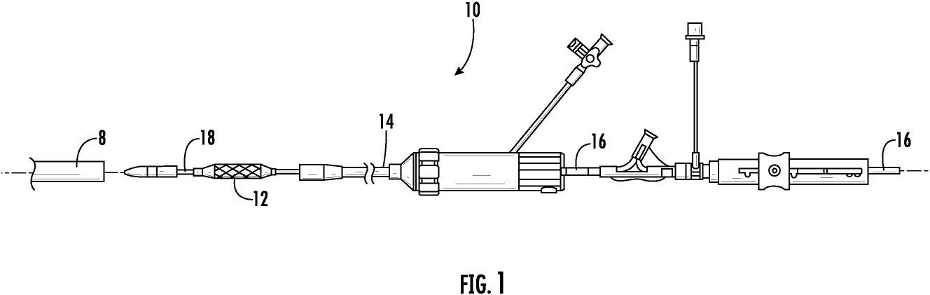

FIG. 1 illustrates an exemplary sheath 8 in use with a representative delivery apparatus 10, for delivering an implant 12, or other type of implantable, to a patient. The delivery apparatus 10 can include a steerable guide catheter 14 (also referred to as a flex catheter) and a balloon catheter 16 extending through the guide catheter 14. The guide catheter 14 and the balloon catheter 16 in the illustrated example are adapted to slide longitudinally relative to each other to facilitate delivery and positioning of the implant 12 at an implantation site in a patient’s body, as described in detail below. The sheath 8 is an elongate, expandable tube that can include a hemostasis valve at the proximal end of the sheath to stop blood leakage.

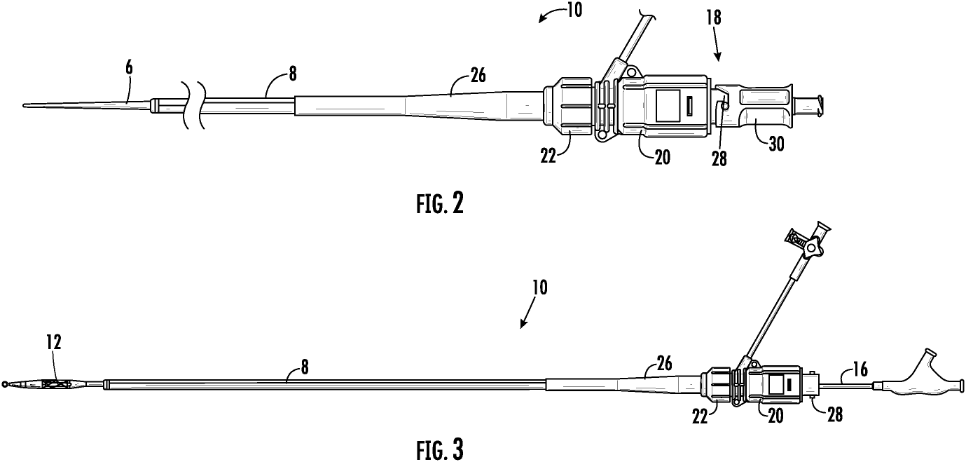

FIG. 2 illustrates the sheath 8 of FIG. 1 including a sheath locking system 18 which prevents axial and rotational translation of the introducer 6 with respect to the sheath 8. The sheath locking system 18 keeps the introducer 6 fixed with respect to the sheath 8 during insertion without requiring a physician or technician to hold the introducer 6 and the sheath 8 in place at the distal end. The sheath locking system 18 includes a locking sleeve 28 coupled to the sheath 8 via sheath hub 20 and an introducer locking hub 30 and introducer 6. The locking sleeve 28 engages the introducer locking hub 30 and is moveable between a locked and unlocked position, thereby fixing the position of the introducer 6 and the sheath 8 and preventing movement therebetween during insertion. As will be described in more detail below, the sheath locking system 18 keeps the introducer 6 from separating from the sheath 8 and prevents gaps from forming that can cause patient abrasions and unintended fluid flow between the introducer 6 and the sheath 8 during insertion.

FIGS. 2, 5A-5B and 6, and illustrate the sheath locking sleeve 28 coupling the introducer locking hub 30 to the sheath hub 20. As will be described in more detail below, the locking sleeve 28 includes a guide 31 that engages a locking channel 38 provided on the introducer locking hub 30. The guide 31 moves within the locking channel 38 between an unlocked position, where the sheath locking sleeve 28 is rotationally and axially movable with respect to the introducer locking hub 30, and a locked position (FIG. 2), where the locking sleeve 28 is axially fixed with respect to the introducer locking hub 30.

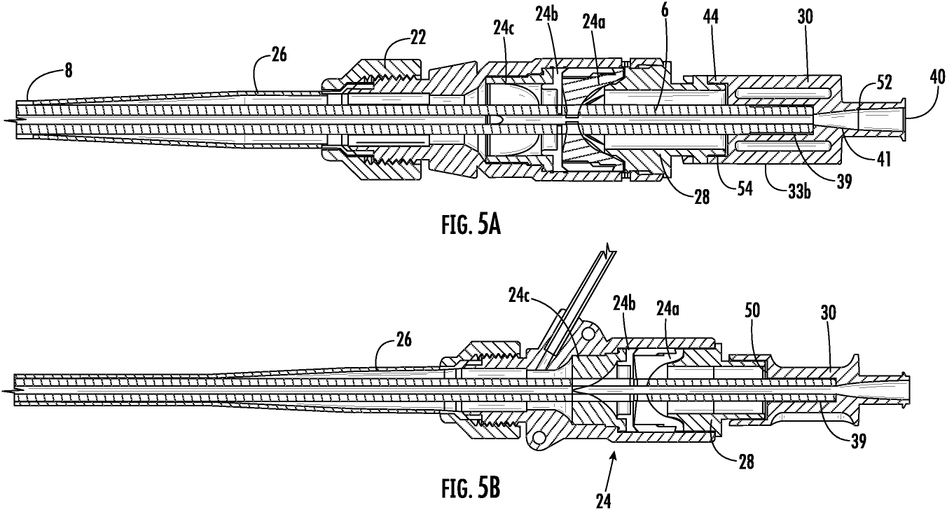

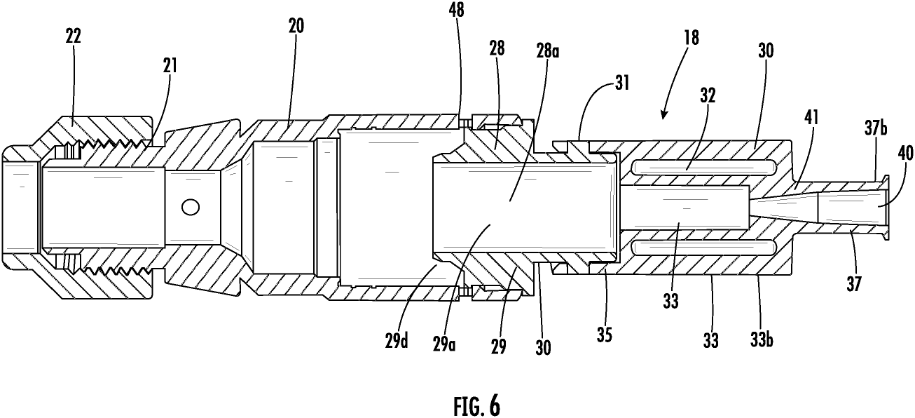

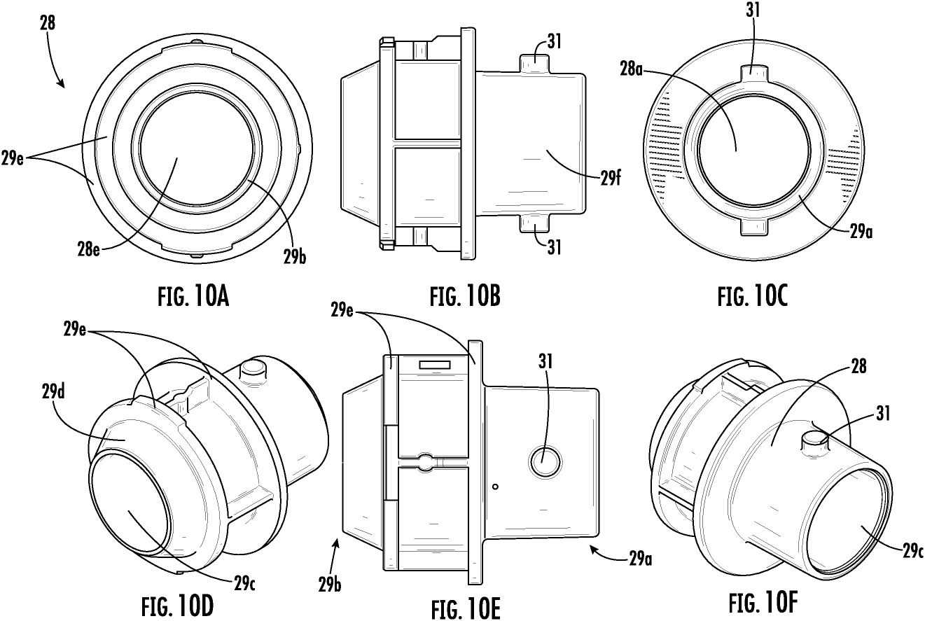

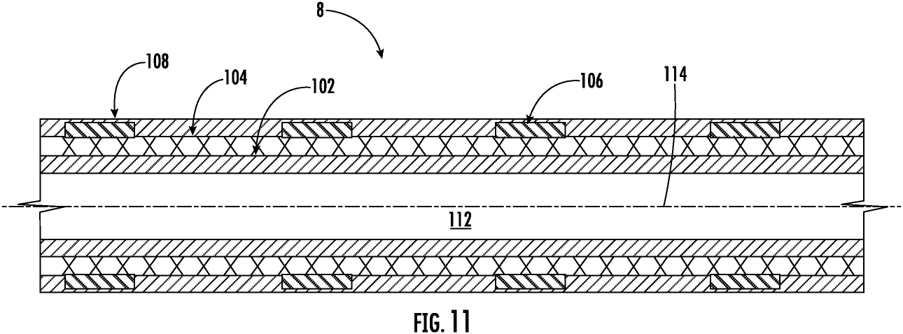

The locking sleeve 28 is illustrated, for example, in FIGS. 10A-10F. The locking sleeve 28 includes an elongated sleeve body 29 with a central lumen 28a extending longitudinally between the proximal end 29a and distal end 29b of the sleeve body 29. As provided in FIG. 6, the central lumen 28a defines a generally cylindrical inner surface 29c of the sheath locking sleeve 28. The central lumen 28a has a diameter of at least 0.3”. In some examples, the diameter ranges between 0.3” and 0.6”. Preferably, the diameter is about 0.40”. The distal end 29b of the sleeve body 29 also has a frustoconical outer surface 29d that tapers about the distal end 29b to help with positioning the locking sleeve 28 within the sheath hub 20 and abutting the seal assembly 24 (FIGS. 5B and 5B). The locking sleeve 28 also has a plurality of interface diameters 29e that extend radially from the outer surface 29d of the sleeve body 29 around (all or a portion of) the circumference of the locking sleeve 28. As illustrated in FIG. 5A, these interface diameters 29e are sized and configured to engage corresponding recesses and/or slots 48 provided in the sheath hub 20 for securing the locking sleeve 28 to the sheath hub 20.

The locking sleeve 28 includes a guide 31 projecting from the outer surface 29f of the locking sleeve 28. The guide 31 engages a corresponding shaped locking channel 38 in the introducer locking hub 30. The guide 31 extends radially from the outer surface 29f and at least partially around the circumference of the outer surface 29f. As provided in FIG. 6, the top surface of the guide 31 does not extend beyond the outer surface of the introducer locking hub 30 when the sheath locking sleeve 28 and the introducer locking hub 30 are coupled. For example, the height of the guide 31 corresponds to the wall thickness of the introducer locking hub 30 proximate the guide when the sheath locking sleeve 28 and the introducer locking hub 30 are coupled. In another example, the top surface of the guide 31 is recessed with respect to the outer surface of the introducer locking hub 30. That is, the height of the guide is less than the wall thickness of the introducer locking hub 30. In other examples, the height of the guide 31 is greater than a wall thickness of the introducer locking hub 30 such that the top surface of the guide 31 extends beyond the outer surface of the introducer locking hub 30 when the sheath locking sleeve 28 and the introducer locking hub 30 are coupled. In some examples, the height/axial length of the guide 31 is between about 0.050” and about 0.10.” In some examples that height/axial length of the guide 31 is about 0.075”.

As illustrated in FIGS. 10D-10F, the guide 31 is a cylindrically shaped projection. However, it is contemplated that the guide 31 may have any other regular or irregular shape that would facilitate movement of the guide 31 within the locking channel 38 of the introducer locking hub 30. For example, the guide 31 may have an elongated hexagon shape. The guide 31 can have a diameter/width ranging from about 0.05” to about 0.20”. Preferably the guide 31 has a diameter/width of about 0.100”.

In general, the locking sleeve 28 can be formed from polycarbonate, but in other implementations the locking sleeve 28 can be formed from rigid plastic, or any other material suitable for providing a strong locking connector for an introducer 6 (metal, composite, etc.)

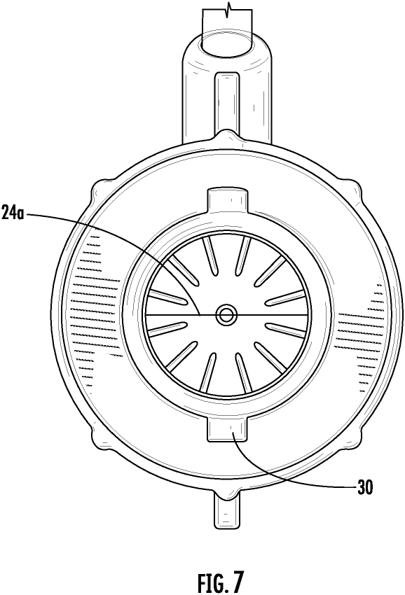

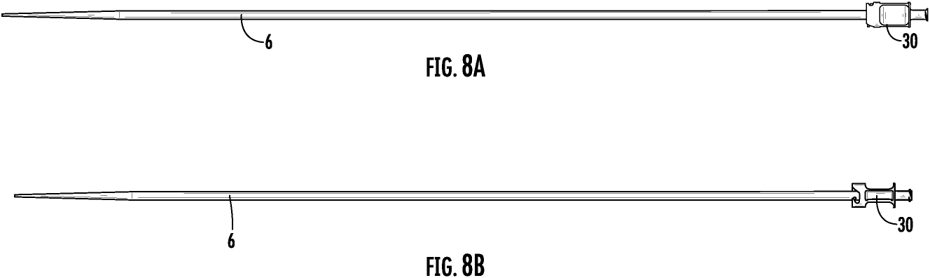

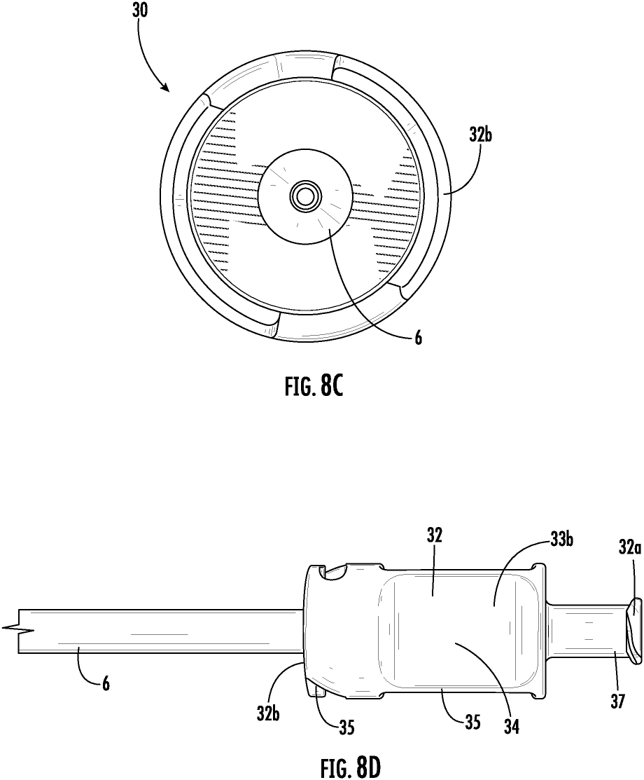

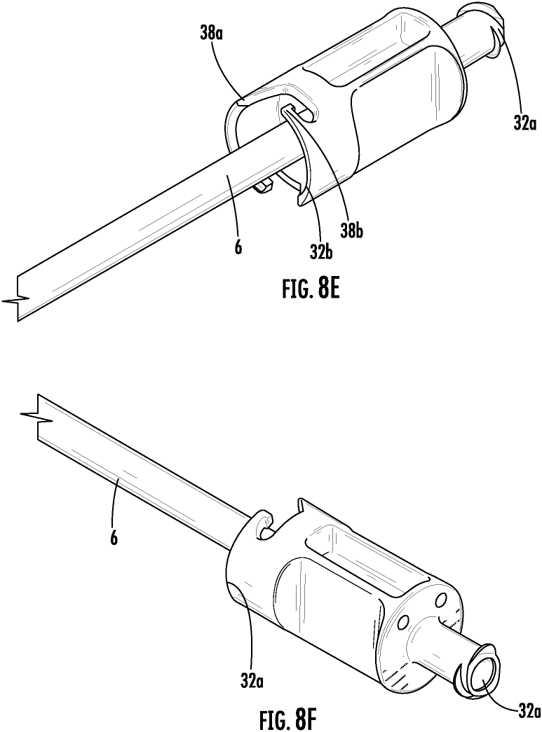

FIGS. 8A-8F illustrate the introducer locking hub 30 with the introducer 6 coupled thereto. Example introducer sheaths are described, for example in U.S. Pat. Nos. 8,690,936 and 8,790,387, 10,639,152 and U.S. Pat. Application Nos. 14/880,106 63/059,764, 63/059,772, the disclosures of which are incorporated herein by reference. As provided in the cross-section views of FIGS. 5A and 5B, the introducer 6 is coupled to the introducer locking hub 30 and extends beyond the distal end of the introducer locking hub 30 body. When coupled to the sheath hub 20, the introducer 6 extends through the central lumen 28a of the sheath locking sleeve 28, the sheath hub 20 and the central lumen of the sheath 8. As will be descried below, the sheath 8 generally comprises a radially expandable tubular structure. Passage of the introducer 6 through the sheath 8 and into a patient’s vessel causes the vessel to radially expand to about the diameter of the sheath 8. That is, the diameter of the central lumen of the sheath 8 is generally about the outer diameter of the introducer 6 such that the introducer 6 provides a mechanism to expand a patient’s vessel to accept the sheath.

As provided in FIGS. 8A-8F, the introducer 6 is formed as an elongate body with a central lumen extending therethrough. As shown in FIGS. 5A and 5B, the central lumen of the introducer is aligned with the central lumens of the introducer locking hub 30, the sheath hub 20 and the sheath 8. The introducer 6 is received within a recessed opening 39 provided on an interior surface of the introducer locking hub 30, the recessed opening 39 axially aligned with the central lumen 45 of the introducer locking hub 30. The introducer 6 is coupled to the introducer locking hub 30 at the recessed opening 39. In an example system, the introducer 6 has a diameter corresponding to or less than the diameter of the recessed opening 39. In some examples, the introducer 6 is fixedly coupled to the introducer locking hub 30 at the recessed opening 39. For example, the introducer 6 is coupled to the recessed opening 39 of the introducer locking hub 30 by at least one of a press fit, an interference fit, a snap fit, a mechanical fastener, a chemical fastener (e.g., an adhesive), a weld, a thermal process, and/or any other suitable coupling process known in the art.

As described above, the introducer 6 has a central lumen that aligns with the central lumen 45 of the introducer locking hub 30. This joined lumen allows for the passage of surgical equipment and/or medical devices to the treatment site. In an example system, and as provided in FIGS. 5A and 5B, the central lumen of the introducer 6 has a diameter corresponding to at least a portion of the diameter of the central lumen 45 of the introducer locking hub 30. In general, the corresponding diameter portion is adjacent the distal end of the central lumen 45. In other examples, the diameter of the central lumen 45 at the distal end of the introducer locking hub 30 is slightly larger than the diameter of the central lumen passing through the introducer 6. The central lumen 45 can also a decreasing tapered portion 41 between the proximal end and the distal end of the introducer locking hub 30 (see FIG. 6). The corresponding diameter portion and decreasing tapered portion 41 allows for smooth transition and delivery of surgical equipment and/or medical device through the introducer locking hub 30 and into the central lumen of the introducer 6.

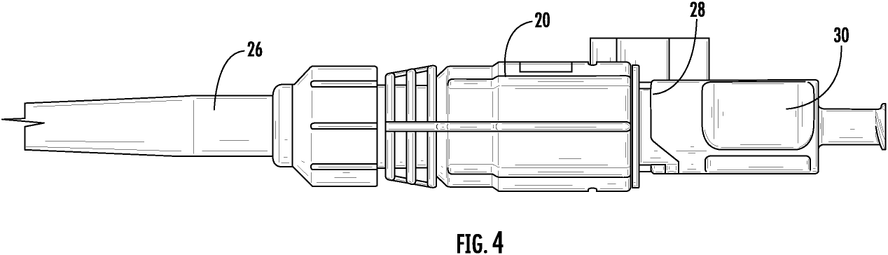

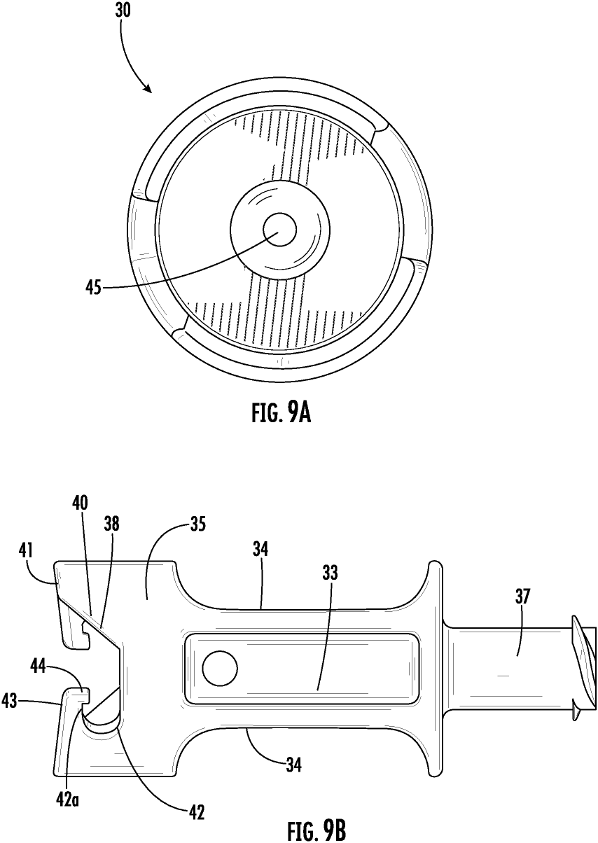

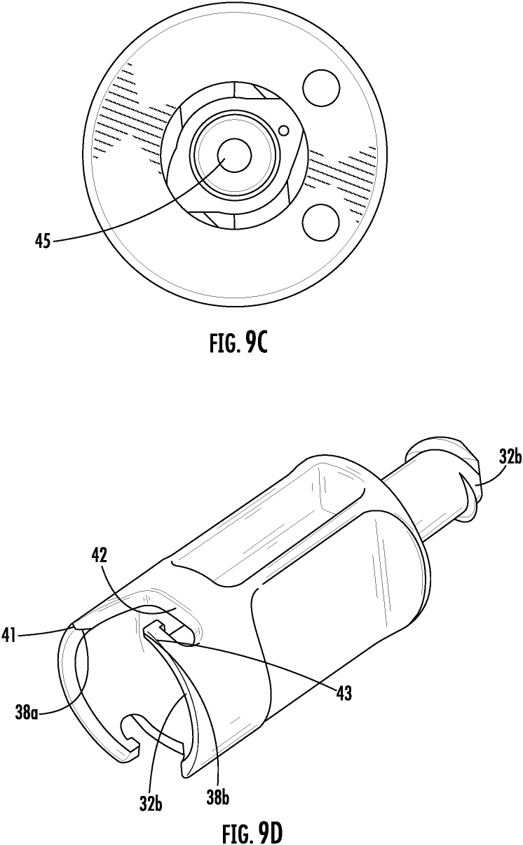

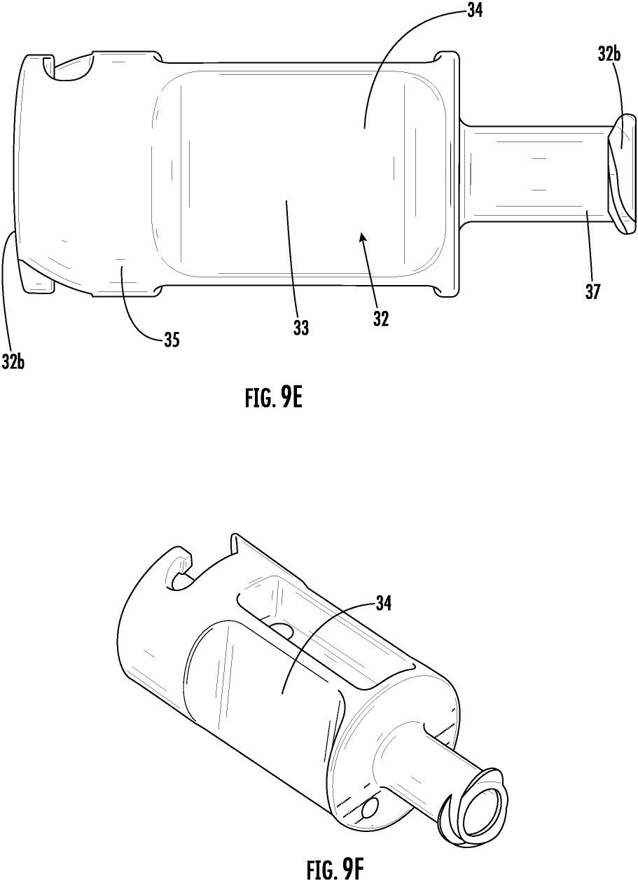

FIGS. 2-6 illustrate the introducer locking hub 30 coupled to the locking sleeve 28. FIGS. 8A-8F provide the introducer locking hub 30 coupled to the introducer 6. FIGS. 9A-9F provide multiple views of the introducer locking hub 30. As described above, the introducer 6 is fixedly coupled to the introducer locking hub 30, and the introducer locking hub 30 couples with the locking sleeve 28 to fix the position the introducer 6 (axially and rotationally) with respect to the locking sleeve 28/sheath 8.

The introducer locking hub 30 includes a hub body 32 having a proximal end 32a and a distal end 32b and defining a central lumen 45 extending therethrough. The hub body 32 has a first (middle) portion 33, a second (distal) portion 35 which extends distally from the first portion 33 and a third (proximal) portion 37 which extends proximally from the first portion 33. The first portion 33 includes the cylindrically-shaped recessed opening 39 for receiving and retaining the introducer 6 and an outer surface 33b. In some examples, the recessed opening 39 has a diameter ranging between 0.15” and about 0.25”. In some examples, the recessed opening 39 has a diameter ranging between 0.17” and about 0.20”. In some examples, the recessed opening has a diameter of about 0.194”.

The third (proximal) portion 37 of the introducer locking hub 30 includes the decreasing tapered portion 41 of the central lumen 45. The decreasing taper portion 41 defining a frustoconical shape with decreasing taper/diameter from the proximal to the distal end of the sheath. It is contemplated that the tapered portion 41 has a minimum diameter of about 0.007” and a maximum diameter of about 0.194”.

As illustrated in FIGS. 5A-5B, when coupled the central lumen 28a of the locking sleeve 28 is aligned with the central lumen 45 of the introducer locking hub 30. In some examples, the central lumen 28a of the locking sleeve 28 is coaxial with the central lumen 45 of the introducer locking hub 30. When coupled, the proximal end of the locking sleeve 28 is received within the central lumen 45 of the introducer locking hub 30. The proximal end surface of the locking sleeve 28 is adjacent a shoulder 50 provided on an inner surface of the central lumen 45 of the introducer locking hub 30. As illustrated in FIG. 5A and5B, the central lumen 45 of the introducer locking hub 30 includes a first portion 52 having a first diameter adjacent the proximal end of the introducer locking hub 30, and a second portion 54 having a second, larger, diameter adjacent the distal end of the introducer locking hub 30. The recessed opening 39 can be consider either a component of the first portion 52 of the central lumen 45, or a separate component of the central lumen 45 locate between the first (proximal) portion 52 and the second (distal) portion 54. When the locking sleeve 28 and introducer locking hub 30 are coupled, at least a portion of the sleeve body 29 of the sheath locking sleeve 28 is received within the second portion 54 (larger portion) of the central lumen 45 of the introducer locking hub 30. The central lumen 28a of the sheath locking sleeve 28 is aligned with the central lumen 45 of the introducer locking hub 30 such that they are co-axial and form a smooth inner surface along the combined central lumens of the introducer locking hub 30 and the sheath locking sleeve 28.

As described generally above, the locking sleeve 28 couples to the introducer locking hub 30 via engagement between the guide 31 on the locking sleeve 28 and the locking channel 38 provided in the introducer locking hub 30. As provided in FIGS. 9A-9F, the introducer locking hub 30 includes two locking channels 38. However, it is contemplated that the introducer locking hub 30 can include one locking channel 38 or more than two locking channels 38. The locking channel 38 can be is formed a recess or groove in a surface of the introducer locking hub 30, as a slotted opening, a clip, or as any other feature capable of receiving and securing the guide 31 projecting from the outer surface of the locking sleeve 28 with the introducer locking hub 30 . Illustrated in FIG. 9B, the locking channels 38 provide an interface to secure the sheath locking sleeve 28 to the introducer locking hub 30 and ensure a fixed axial position between the introducer 6 and the sheath 8.

The locking channel 38 is formed on the distal end of the introducer locking hub 30. The locking channel 38 includes an opening on the distal end surface that leads to an angled guide portion 40 that transitions to a locking portion 42. The guide portion 40 is configured to direct the guide 31 of the locking sleeve 28 in an axial and circumferential direction along the side wall of the guide portion 40 towards the locking portion 42 upon rotation of the introducer locking hub 30 and/or the sheath locking sleeve 28. The locking portion 42 is configured to securely engage the guide 31, fixing the axial position of the introducer locking hub 30 with respect to the sheath locking sleeve 28. As illustrated in FIG. 9B, the guide portion 40 of the locking channel 38 extends from the distal end of the introducer locking hub 30 axially towards the proximal end of the introducer locking hub 30 and circumferentially around the introducer locking hub 30. For example, the guide portion 40 of the locking channel 38 can be described as extending helically around/along a length of the introducer locking hub 30 or on an angle from the distal end of the introducer locking hub 30.

As illustrated in FIGS. 9B and 9D, the locking portion 42 of the locking channel 38 extends at an angle from the end of the guide portion 40. As provided in FIG. 9B, the angle between the centerline of the guide portion 40 and the centerline of the locking portion 42 is greater than 90-degrees. In another example, the angle between the centerline of the guide portion 40 and the centerline of the locking portion 42 is about 120-degrees. In an example system, the locking portion 42 extends around a portion of the circumference of the introducer locking hub 30. The locking portion 42 can extend parallel to the distal end of the introducer locking hub 30. In an example system, the length of the guide portion 40 (measured along its centerline) is greater than a length of the locking portion 42 (measured along its centerline). In another example, the length of the guide portion 40 equals or is less than a length of the locking portion 42.

The locking portion 42 can include a catch 44 for securing the guide 31 within the locking portion 42 of the locking channel 38 and forming a partial barrier for the guide 31 within the locking portion 42. As illustrated in FIG. 9B, the catch 44 includes a projection that extends from a side wall 42a of the locking portion 42 and releasably secures the guide 31 within the locking channel 38. The catch 44 extends from the side wall 42a of the locking portion 42 in a proximal direction towards the center line of the locking portion 42 and has a height sufficient to retain the guide 31 between the catch 44 and the end of the locking portion 42.

The distal end surface 32b of the introducer locking hub 30 can include features for biasing the guide 31 towards the locking channel 38. For example, the distal end of the introducer locking hub 30 can include a tapered surface angled toward an opening of the locking channel 38. As illustrated in FIG. 9B, the distal end 32b of the introducer locking hub 30 includes a first tapered surface (decreasing tapered portion 41) angled towards a leading edge of the opening of the locking channel 38 and a second tapered surface 43 angled towards the trailing edge of the opening of the locking channel 38.

In use, engagement between the guide 31 and the guide portion 40 of the locking channel 38 is configured to bias the locking sleeve 28 in a proximal axial direction toward the proximal end of the introducer locking hub 30 (towards a locked position) when the sheath locking sleeve 28 is rotated in a first axial direction. In this direction the guide 31 advances toward the locking portion 42 of the locking channel 38 into the locked position. Alternatively, engagement between the guide 31 and the locking portion 42 of the locking channel 38 is configured to bias the locking sleeve 28 in a distal axial direction toward the distal end of the introducer locking hub 30 (towards an unlocked position) when the sheath locking sleeve 28 is rotated in a second (opposite) axial direction. In the second direction, the guide 31 advances away from the locking portion 42 of the locking channel 38, to the unlocked position. When the guide 31 is in the locked position and retained with by locking portion 42 by catch 44, rotation in the second direction causes the guide 31 to bias against the catch 44 overcoming the oppositional forces of the catch 44, and moving the guide 31 from the locked to the unlocked position.

As illustrated in FIGS. 8A-9F, the outer surface 33b of the introducer locking hub body 32 includes gripping features and/or surfaces for a physician or technician to use when manipulating the introducer locking hub 30. As provided in FIG. 9B, the introducer locking hub body 32 can include a two recessed gripping surfaces 34 on opposite sides of the longitudinal axis of the introducer locking hub 30. When the introducer locking hub 30 is viewed from the side, the gripping surfaces 34 define a dog-bone/barbell shape to the hub body 32, i.e., a shape having a smaller diameter/width center portion and larger diameter/width end portions. In an example system, the gripping surfaces 34 are provided along at least 40% of the length of the introducer locking hub body 32. In another example, the gripping surfaces 34 are provided along at least 50% of the length of the introducer locking hub body 32.

In general, the introducer locking hub 30 can be formed from polycarbonate, but in other implementations the introducer locking hub 30 can be formed from rigid plastic, or any other material suitable for providing a locking mechanism for an introducer 6 (metal, composite, etc.).

FIGS. 2-6 illustrate an example sheath hub 20. As described above, the sheath 8 is coupled to the sheath hub 20 which in turn is removably coupled to the locking sleeve 28. The sheath hub 20 provides a housing for necessary seal assemblies and an access point for a secondary lumen (e.g., fluid lumen) in fluid communication with the central lumen of the sheath hub 20.

The sheath hub 20 further has receiving slots 48. The receiving slots 48 are openings which extend around a portion of the diameter of the sheath hub 20 and are sized and configured to accept the interface diameters 29e. Coupling between the receiving slots 48 and the interface diameters 29e of the locking sleeve 28 axially and rotationally fixes the locking sleeve 28 and the sheath hub 20 relative to each other.

The distal end of the sheath hub 20 includes threads 21 for coupling to a threaded sheath hub cap 22. The sheath 8 is provided between the sheath hub 20 and the sheath hub cap 22 such that coupling the sheath hub cap 22 to the sheath hub 20 fixes the sheath 8 to the sheath hub 20. The sheath hub cap 22 is a cylindrical cap having a cap body having a proximal end and a distal end and defining a central lumen extending longitudinally between the proximal end and the distal end. The sheath hub cap 22 has a larger diameter at its proximal end than at its distal end.

As described above and as shown in FIGS. 5A and 5B and the distal end view of the locking sleeve 28 shown in FIG. 7, a seal assembly 24 is included in sheath hub 20. The seal assembly 24 includes proximal seal 24a, intermediate seal 24b, and distal seal 24c. When assembled, the introducer 6 passes through the seal assembly and extends distal of the sheath 8. The proximal seal 24a, the intermediate seal 24b, and the distal seal 24c are each formed to prevent unwanted fluid from advancing in the proximal direction through the sheath hub 20 and proximal of the seal assembly 24. They are each openable and closable to provide pressure variation to affect the desired fluid flow from a physician or technician.

As illustrated in FIG. 2, the sheath 8 includes a seal tube 26/strain relief portion. The seal tube 26 is coupled to the distal end of the sheath hub 20 and creates a smooth transition surface between the sheath 8 and the sheath hub 20. The frustoconical seal tube 26 body has a proximal end and a distal end and a central lumen extending longitudinally therethrough. The seal tube 26 tapers from the proximal end to the distal end such that the diameter of the seal tube 26 at the proximal end is greater than the diameter of the seal tube 26 at the distal end of the seal tube 26.

A method for delivering a prosthetic device to a procedure site such that axial movement between the introducer 6 and the sheath 8 is eliminated is described below. Preventing gapping between the introducer 6 and the sheath 8 during insertion reduces the risk of trauma to the patient’s vasculature. FIG. 2 shows the example device for delivering the prosthetic device.

The method includes providing an introducer locking hub 30 having an elongated introducer 6 coupled to the hub body 32 of the introducer locking hub 30. As described above the introducer locking hub 30 including a locking channel 38 disposed in the hub body 32. The sheath locking sleeve 28 is advanced to a position adjacent a distal end of the introducer locking hub 30 such that a guide 31 projecting from an outer surface of the sheath locking sleeve 28 is received within the opening to the locking channel 38. Advancing the sheath locking sleeve 28 to a position adjacent the distal end of the introducer locking hub 30 also includes advancing the introducer 6 axially within the central lumen of the expandable delivery sheath 8.

The introducer locking hub 30 is then rotated in a first direction with respect to the locking sleeve 28 to move the guide 31 along the locking channel 38 into a locked position. In particular, moving the guide 31 into the locked position includes rotating the introducer locking hub 30 to move the guide 31 along a guide portion 40 of the locking channel 38 toward a locking portion 42. Further rotation of the introducer locking hub 30 directs the guide 31 into the locking portion 42 of the locking channel 38, the locking portion 42 configured to securely engage the guide 31 and fix the axial position of the introducer locking hub 30 with respect to the sheath locking sleeve 28. Where the locking channel 38 includes a catch 44, rotation of the introducer locking hub 30 in the first direction causes the guide 31 to overcome the bias force of the catch 44 and advance the guide 31 beyond the catch 44 into the locking portion 42, where the catch 44 secures the guide 31 within the locking portion 42 thereby fixing the axial location of the sheath 8 with respect to the introducer 6.

The coupled sheath 8 and introducer 6 are then inserted, at least partially, into the vasculature of the patient.

Once positioned, the introducer locking hub 30 is rotated in a second, opposite, direction with respect to the locking sleeve 28. Rotating the introducer locking hub 30 in the second direction causes the guide 31 to slide along the locking channel 38, from the locking portion 42 toward the guide portion 40. In particular, rotating of the introducer locking hub 30 in the second direction directs the guide 31 out of the locking portion 42 of the locking channel 38 and through the guide portion 40 and releases the introducer locking hub 30 from the sheath locking sleeve 28. Where the locking channel 38 includes a catch 44, rotation of the introducer locking hub 30 in the second direction causes the guide 31 to overcome the bias force of the catch 44 and advance from the locking portion 42 to the guide portion 40 of the locking channel 38. As a result, the guide 31 slides out of the locking channel 38 into the unlocked position.

The introducer locking hub 30 is then disengaged from the locking sleeve 28 and the introducer 6 is withdrawn from the central lumen of the sheath 8. With the central lumen of the sheath 8 clear, the medical device (e.g., implant 12) is advanced through the central lumen of the sheath 8. The medical device (implant 12) is delivered to the procedure site via the central lumen of the sheath 8.

A method of securing a delivery sheath to an introducer in a device for prosthetic heart valve delivery device is disclosed herein. The method comprises providing an introducer locking hub 30 having an elongated introducer 6 coupled thereto and including a locking channel 28 disposed in the hub body 32. The sheath locking sleeve 28 is advanced to a position adjacent a distal end of the introducer locking hub 30 such that a guide 31 projecting from an outer surface of the sheath locking sleeve 28 is received within an opening of the locking channel 38. Advancing the sheath locking sleeve 28 to a position adjacent the distal end of the introducer locking hub 30 also includes advancing the introducer 6 axially within the central lumen of the expandable delivery sheath 8.

The introducer locking hub 30 is then rotated in a first direction with respect to the locking sleeve 28 to move the guide 31 along the locking channel 38 into the locked position. In particular, moving the guide 31 into the locked position includes rotating the introducer locking hub 30 to move the guide 31 along a guide portion 40 of the locking channel 38 toward a locking portion 42. Further rotation of the introducer locking hub 30 directs the guide 31 into the locking portion 42 of the locking channel 38, the locking portion 42 configured to securely engage the guide 31 and fix the axial position of the introducer locking hub 30 with respect to the sheath locking sleeve 28. Where the locking channel 38 includes a catch 44, rotation of the introducer locking hub 30 in the first direction causes the guide 31 to overcome the bias force of the catch 44 and advance the guide 31 beyond the catch 44 into the locking portion 42, where the catch 44 secures the guide 31 within the locking portion 42 thereby fixing the axial location of the sheath 8 with respect to the introducer 6.

To unlock the introducer locking hub 30 from the locking sleeve 28, the introducer locking hub 30 is rotated in a second, opposite, direction with respect to the locking sleeve 28. Rotating the introducer locking hub 30 in the second direction causes the guide 31 to side along the locking channel 38, from the locking portion 42 toward the guide portion 40. In particular, rotating of the introducer locking hub 30 in the second direction directs the guide 31 out of the locking portion 42 of the locking channel 38 and through the guide portion 40 to release the introducer locking hub 30 from the sheath locking sleeve 28. Where the locking channel 38 includes a catch 44, rotation of the introducer locking hub 30 in the second direction causes the guide 31 to overcome the bias force of the catch 44 and advance from the locking portion 42 to the guide portion 40 of the locking channel 38. As a result, the guide 31 slides out of the locking channel 38 into the unlocked position.

The introducer locking hub 30 is then disengaged from the locking sleeve 28 and the introducer 6 can be withdrawn from the central lumen of the sheath 8.

In some procedures, a curved approach to the treatment site is desirable. For example, during a transseptal approach for mitral valve replacement/repair. Mitral valve diseases are among the most prevalent valvular heart diseases and necessitate surgical procedures for the repair or replacement of this valve. Conventional left atriotomy is the standard approach for most surgeons. However, the transseptal approach can confer better exposure to the mitral valve in cases where the left atrium is small, where there are adhesions caused by previous procedures, where there are associated operations requiring right atriotomy, and where there is beating heart surgery.

In the transseptal approach, the right atrium is opened and a longitudinal incision (approximately 4 cm) made in the middle of the foramen ovalis on the intra-atrial septum. The septal edges are then pulled in order to expose the mitral valve fully. Conventionally a dilator and/or shunt is required to expand the opening, a shunt can also be used to close the opening and provide an access point for future procedures. However, a major drawback of the transseptal approach surrounds the risk associated with expanding the incision, maintaining the expanded opening the foramen ovalis and the use of a shunt for closing the opening. The sheath of the present disclosure allows for the local and temporary expansion of the incision site only during delivery of the prosthetic device through the incision site.

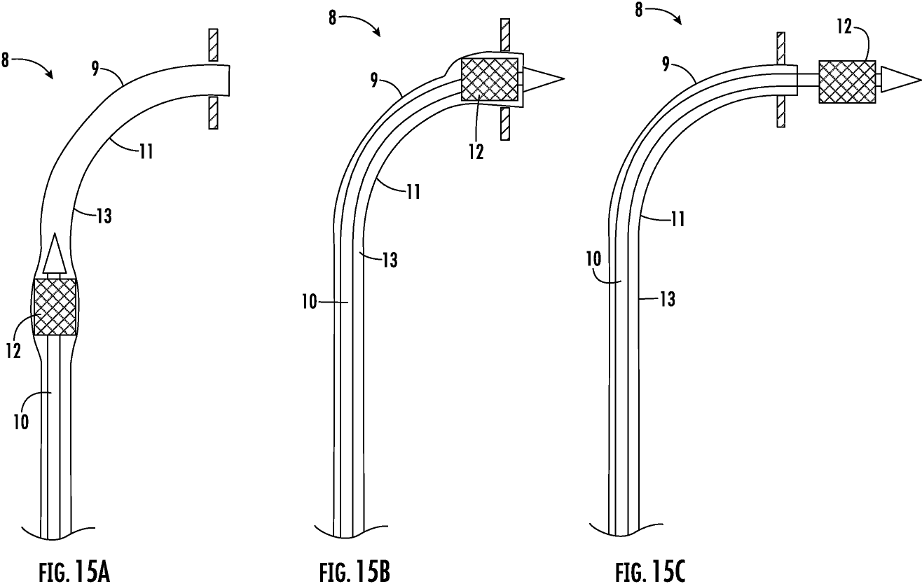

As described above, the sheath assembly 8 and introducer 6 can be used to introduce the delivery apparatus 10 and the prosthetic device (e.g., implant 12) into a patient’s body. Disclosed herein is a system and method for bending/curving the distal end of the sheath 8 to allow for a curved approach to the treatment site. As illustrated in FIG. 3, the introducer device/sheath assembly 8 can comprise the sheath hub 20 at a proximal end of the device and an expandable sheath 8 extending distally from the sheath hub 20. The sheath hub 20 can function as a handle for the device. The expandable sheath 8 has a central lumen to guide passage of the delivery apparatus for the medical device/prosthetic heart valve. In an alternative examples, the introducer device/sheath assembly need not include the sheath hub 20. For example, the sheath 8 can be an integral part of a component of the sheath assembly, such as the guide catheter. As described above, the sheath 8 can have a natural, unexpanded outer diameter that will expand locally upon passage of the medical device. In certain examples, the expandable sheath 8 can comprise a plurality of coaxial layers extending along at least a portion of the length of the sheath 8. Example expandable sheaths are described, for example, in U.S. Pat. Application No. 16/378,417, entitled “Expandable Sheath,” and U.S. Provisional Pat. Application No. 62/912,569, entitled “Expandable Sheath,” the disclosures of which are herein incorporated by reference. The structure of the coaxial layers is described in more detail below with respect to FIGS. 11-14. The structure facilitating the curving/bending of the distal end of the sheath 8 is described in reference to FIGS. 15A-15C.

In general, the sheath 8 can include a first tubular layer (inner layer 102) and a resilient elastic tubular second layer 104 radially outward of the inner layer 102, the elastic second layer 104 being configured to apply radial force to the inner layer 102. When a medical device (implant 12) is passed through the sheath 8 (FIG. 15A), the diameter of the sheath 8 temporarily and locally expands from an initial diameter to an expanded diameter around the medical device (implant 12). The sheath 8 resiliently returns to the initial diameter by radial force applied by the elastic second layer 104 upon passage of the medical device (e.g., implant 12). As provided in FIGS. 15A-15C, the distal tip 9 of the sheath 8 is configured to bend in a direction away from a longitudinal axis of the sheath 8. To facilitate bending, the distal tip 9 can be constructed from a more flexible foramen ovalis than the remaining portion of the sheath 8. That is, the distal tip 9 can be constructed from a material having a lower stiffness than a material of the remaining portion of the sheath 8. The distal tip 9 can also include some feature or treatment that would encourage bending. For example, slits or groove could be cut into the outer surface of the sheath 8 along the distal tip 9. The slits/grove would create a weakened portion and voids along the length, encouraging the sheath 8 to curve along this portion. Various methods of bending the distal tip 9 are described below.

The distal tip 9 can be integrally formed with the remaining portion of the sheath 8. That is, the distal tip 9 can be constructed from the same coaxial layered material structure as the remaining portion of the sheath 8. In another example, the distal tip 9 can be coupled to the distal end of the sheath 8. For example, the distal tip 9 can be constructed as a separate working channel, having different material properties than the sheath 8, that is coupled to the distal end of the sheath 8. In these examples, the distal tip 9 can be fixedly coupled to the distal end of the sheath 8. For example, the distal tip 9 can be coupled to the remaining portion of the sheath 8 by a mechanical fastener, a chemical fastener, a thermal process, or suitable means for coupling the distal tip 9 to the sheath 8. Similar to the sheath 8, the distal tip 9 temporarily and locally expands from an initial diameter to an expanded diameter around the medical device (e.g., implant 12). For example, when the medical device is passed through the central lumen of the distal tip 9, the diameter of the distal tip 9 expands from an initial tip diameter to an expanded tip diameter around the medical device (implant 12). After, the distal tip 9 resiliently returns to the initial tip diameter upon passage of the medical device. Similar to the sheath 8, an elastic layer can be provided over the distal tip and a radial force applied by the elastic layer urges the distal tip 9 to return to the initial diameter upon passage of the medical device (implant 12).

In an example system, the sheath 8 can include a pull wire to facilitate bending of the distal tip 9. As illustrated in FIGS. 15A-15C, the sheath 8 (and distal tip 9) can include a pull wire lumen 11 extending from the distal tip 9 of the sheath and a proximal end of the sheath 8 and a pull wire 13 extending therethrough. The pull wire lumen 11 can be embedded within the wall thickness of the sheath 8. In another example, the pull wire lumen 11 is provided along the central lumen of the sheath 8. In a further example, the pull wire lumen 11 is provided along the outer surface of the sheath 8. It is further contemplated that the pull wire 13 can extend through the central lumen of the sheath 8 or along the outer surface of the sheath 8. In these examples, a guide feature may be provided along the length of the central lumen and/or outer surface of the sheath 8 to ensure the pull wire 13 does not stray from its intended routing along the sheath 8. Regardless of position (withing wall thickness, within central lumen, exterior of sheath 8), the pull wire lumen 11/pull wire 13 is laterally offset from the longitudinal axis of the sheath 8. In general, the pull wire 13 extends along a side of the sheath 8. A force applied to the pull wire causes the distal tip 9 of the sheath 8 to approximate a curved shape. Tension on the pull wire 13 will curve the distal tip 9 of the sheath 8 in a direction corresponding to the pull wire 13. For example, the pull wire 13 can extend along a first side (e.g., right) of the sheath 8. Tension application to the pull wire 13 will cause the sheath 8 (at the distal tip 9) to curve in a direction corresponding to the first site of the sheath (e.g., curve to the right, away from the longitudinal axis of the sheath 8). Similarly, release of the tension on the pull wire 13 will cause the sheath 8 to straighten and return towards the original straight profile.

The pull wire 13 can be coupled to the distal tip 9 at a coupling point proximate the distal end of the sheath. The pull wire can be coupled at the distal end surface of the sheath 8 or at a location proximate the distal end surface. In another example, the coupling point for the pull wire is offset from the distal end of the sheath 8. The pull wire can be coupled to the sheath 8/pull wire lumen 11 but a mechanical fastener (e.g., an anchor, a clip, a pin) and/or chemical fastener. The pull wire can also be coupled to the sheath/pull wire lumen by a heat treatment process.

In another example (not shown), a curved stylet may be used to curve the distal tip 9 of the sheath 8. For example, a curved stylet may be provided that is movable within the central lumen of the sheath 8. The distal end of the stylet can include a curved portion that, when received within the central lumen of the sheath 8, effects a corresponding curvature of the sheath 8. The stylet can be movable within the sheath 8 to a final position such that the curved portion of the stylet is proximate the distal tip 9 of the sheath 8 and the stylet effects a corresponding curvature of the distal tip 9. In general, the stylet may include a central lumen for receiving/passing over a guidewire or other medical device.

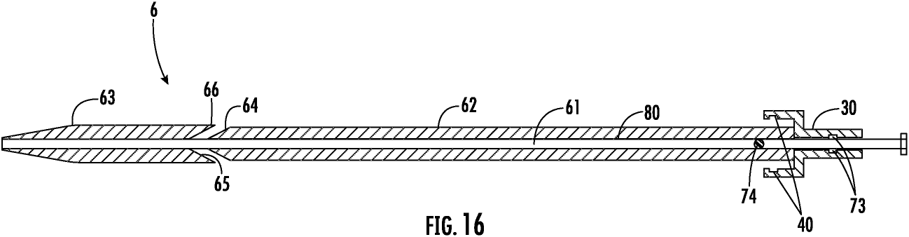

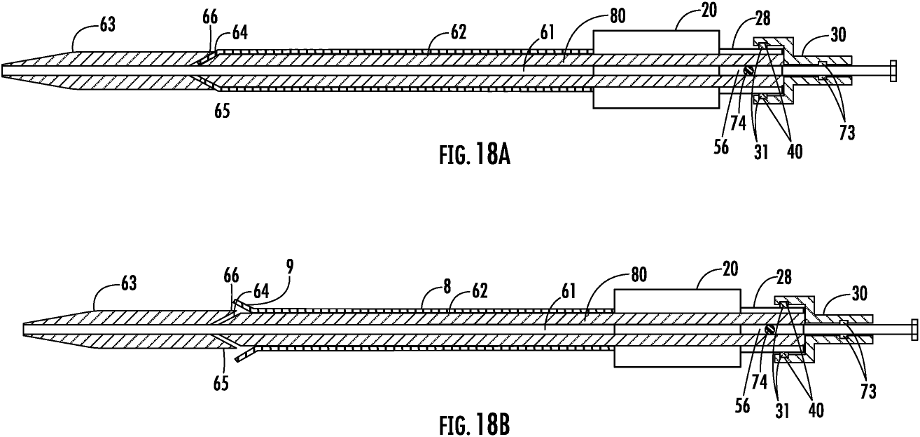

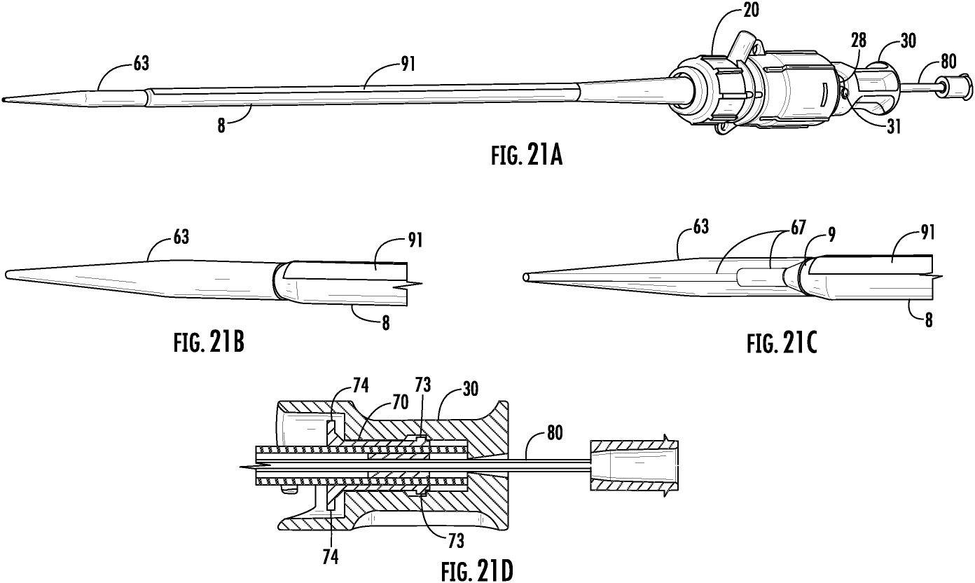

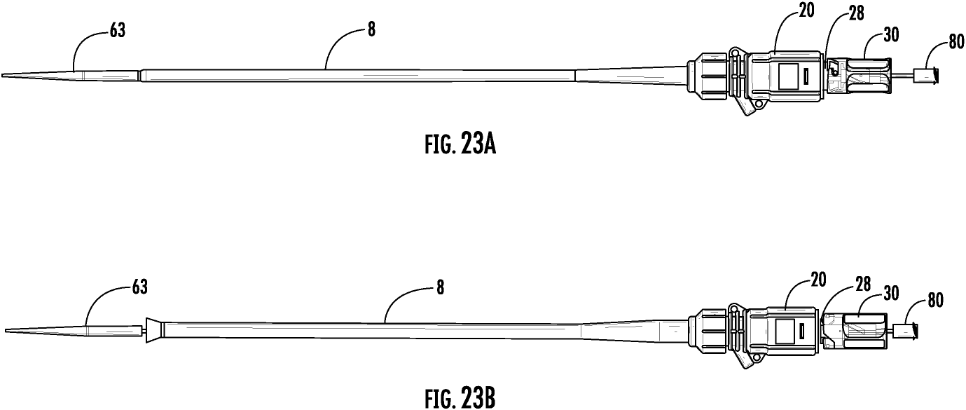

As illustrated in FIG. 16-23B, in another example, the distal tip 9 of the sheath 8 can be retained within the introducer 6 during insertion into the patient’s vasculature. As illustrated in FIGS. 18A and 21A retaining the distal tip 9 of the sheath 8 within the introducer 6 ensures a smooth transition between the end of the sheath 8 and the introducer 6 helping to prevent damage to the patient’s vasculature during insertion. As will be described in more detail below, the introducer 6 can be divided into two, separable, segments that can be used to retain the distal tip 9 of the sheath 8.

FIG. 16-18B provide a schematic representation of an example system including the segmented introducer 6. As described above, the introducer 6 is coupled to the introducer locking hub 30 and includes a central lumen 61 extending therethrough. The introducer 6 defines an elongated tubular body having proximal piece 62 and a separable distal piece 63. A hypotube 80 is disposed within the central lumen 61 of the introducer 6 and coupled to the distal piece 63 of the introducer 6. At least a portion of the hypotube 80 (e.g., the elongated body portion 81) is slidably disposed in the central lumen 61 of the proximal piece 62 of the introducer 6. Accordingly, movement of the hypotube 80 within the central lumen 61 of the proximal piece 62 causes the distal piece 63 of the introducer 6 to move with respect to the proximal piece 62 of the introducer 6. For example, the distal piece 62 of the introducer 6 is movable from a first position (FIG. 18A) adjacent the proximal piece 63 to a second position (FIGS. 16, 18B) spaced from the proximal piece 62.

As illustrated in the transition between FIGS. 18A and 18B, axial movement of the hypotube 80 within the central lumen 61 of the proximal piece 62 of the introducer 6 results in a corresponding axial movement of the distal piece 63 of the introducer 6. When the introducer 6 is in the first position (FIG. 18A), axial movement of the hypotube 80 in a distal direction (e.g., toward the distal end of the hypotube 80/introducer 6) causes the distal piece 63 to move from the first position to/toward the second position and the distal tip 9 of the sheath 8 to release from the introducer 6. Similarly, when the introducer 6 is in the second position (FIGS. 16, 18B), axial movement of the hypotube 80 in a proximal direction (e.g., toward the proximal end of the introducer 6/hypotube 80) causes the distal piece 63 to move from the second position to the first position. It is contemplated that the spacing between the proximal piece 62 and the distal piece 63 can range between about 0.25 inches and about 1.25 inches, between about 0.5 inches and about 1.0 inches, where the spacing is measured along the outer diameter of the introducer 6. In addition to axial movement, rotational movement of the hypotube 80 within the central lumen 61 of the proximal piece 62 of the introducer 6 results in a corresponding rotational movement of the distal piece 63 of the introducer 6. The proximal piece 62 of the introducer 6 is fixedly coupled to the introducer locking hub 30, whereas the distal piece 63 is separable from the proximal piece 62 and coupled to the distal end 81b of hypotube 80. Accordingly, rotational movement/force applied to the hypotube 80 causes the distal piece 63 to rotate independent of the proximal piece 62.

As described above, the introducer 6 is coupled to the introducer locking hub 30 such that the central lumen 61 of the introducer 6 is aligned with the central lumen 40 of the introducer locking hub 30. For example, the central lumen 61 of the introducer 6 may be coaxial with the central lumen 40 of the introducer locking hub 30. The proximal piece 62 of the introducer 6 is fixedly coupled to the introducer locking hub 30. For example, the proximal piece 62 can be coupled to the introducer locking hub 30 by at least one of a press fit, an interference fit, a snap fit, a mechanical fastener, a weld and/or an adhesive. As illustrated in FIGS. 16, 18A-18B and 22A-22B, the proximal piece 62 of the introducer 6 includes a tapered distal end 64. As provided in FIGS. 16, 18A and 21C, at least a portion of the tapered distal end 64 of the proximal piece 62 is received within the central lumen 61 of the distal piece 63 when the introducer 6 is in the first position. The central lumen 61 of the distal piece 63 includes a corresponding tapered surface 65. In another example, the central lumen 61 of the distal piece 63 includes cylindrical opening sized and configured to receive the tapered distal end 64 of the proximal piece 62. It is contemplated, that the distal end of the proximal piece 62 can have any suitable shape to facilitate compression and/or retention of the distal tip 9 of the sheath 8 between the distal end of the proximal piece 62 and the proximal end/interior surface of the distal piece 63.

As illustrated in FIGS. 16, 18A-18B and 21A-22B, the outer diameter of the proximal piece 62 is the same as an outer diameter of the distal piece 63. For example, the outer diameter of the proximal and distal pieces 62, 63 ranges between about 0.131 inches (e.g., 10F) and about 0.236 inches (18F), and preferably about 0.170 inches (e.g., 14F). Similarly, the central lumen 61 diameter of the proximal and distal pieces 62, 63 is generally greater than 0.035 inches (e.g., large enough to accommodate a 0.035 inch diameter guidewire). In another example, the outer diameter of the proximal piece 62 is less than an outer diameter of the distal piece 63 to facilitate layering of the sheath 8 on the outer surface of the introducer and reduce the overall profile of the sheath 8/introducer 6 during insertion in the patient’s vasculature. Similarly, the distal end of the distal piece 63 can include a tapered nosecone to reduce trauma to patient tissue during insertion.

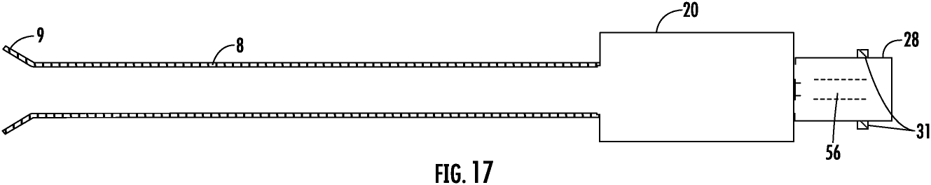

As described above, the introducer 6 is configured such that the distal tip 9 of the sheath 8 can be retained between the proximal and distal pieces 62, 63 of the introducer 6. Similar to the sheath 8 described above, and as illustrated in FIGS. 17, 20, 21A, and 22A, the sheath 8 is coupled to a sheath hub 20. The sheath 8 defines an elongated tubular body with a central lumen extending therethrough. The central lumen of the sheath 8 is aligned with the central lumens of the sheath hub 20 and the introducer locking hub 30.

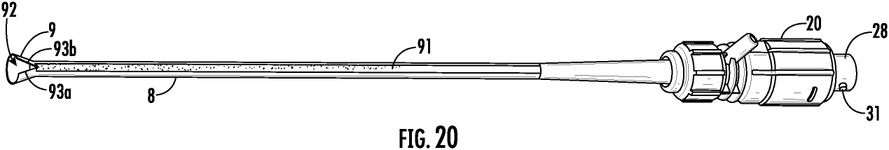

The sheath 8 includes an expandable body portion 91 (e.g., including longitudinally-extending folds or creases that expand in response to a radially outward directed force). The distal tip 9 is provided at the distal end of the body portion 91. As illustrated in FIG. 20, the distal tip 9 is biased in an expanded configuration. The expanded configuration of the distal tip 9 facilitates post insertion needs such as balloon retrieval and retrieval and/or repositioning of the heart valve. Minimization the retrieval force is preferable eases the transition of the valve (and/or balloon) from an expanded state to a crimped/compressed state and passage into the sheath lumen. For example, the distal tip 9 can be comprised of a heat set or shape memory material that is formed to return to the expanded configuration when in a neutral state. In one example, the distal tip 9 includes a polymer or polymer composite material heat set into default to the expanded configuration. In another example, the distal tip 9 (and optionally the body portion 91) includes a shape memory material, e.g., polymer or alloy such as nitinol, that is deformed when cold but returns to its pre-deformed (“remembered”) shape when heated. That is, the shape memory material of the distal tip 9 can be deformed and positioned between the proximal and distal piece 62, 63 of the introducer 6 prior to insertion into the patient’s vasculature. Once inserted (and the proximal and distal pieces 62, 63 separated) the patient’s body temperature causes the shape memory material to return to it’s formed (“remembered”) expanded configuration.

As illustrated in FIGS. 17, 20, and 22A-22B, in the expanded configuration, the outer diameter of the distal end of the distal tip 9 is greater than the diameter of the expandable sheath 8. In general, the outer diameter of the body portion 91 of the unexpanded sheath 8 ranges between about 0.300 inches and about 0.200 inches, particularly where the introducer 6 has a diameter of about 0.184 inches (14F). As provided in FIGS. 17, 20 and 22A-22B, the expanded distal tip 9 can define a frustoconical shape.

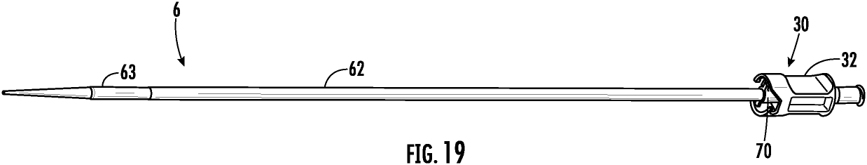

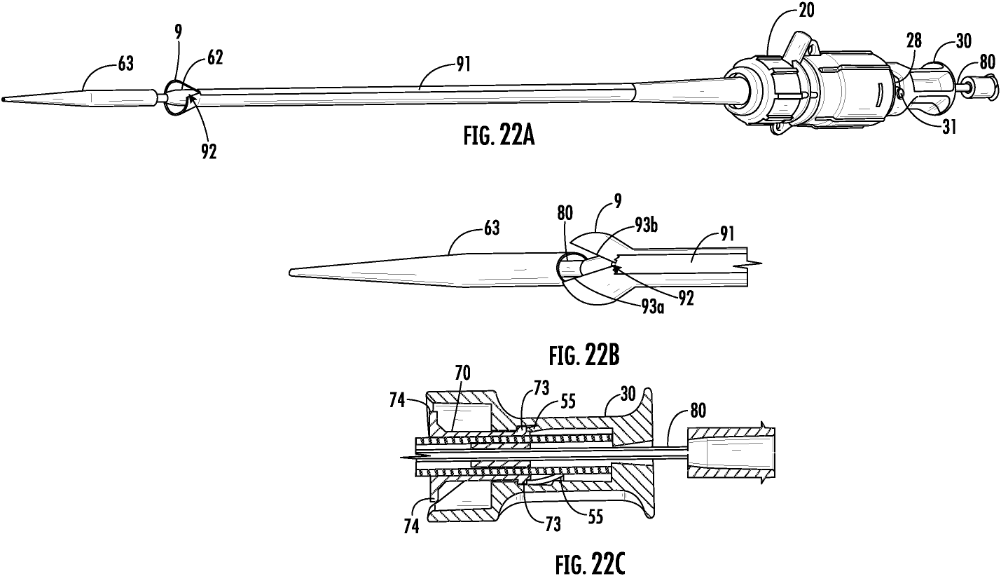

As described above, and illustrated in FIGS. 18A and 19, when the distal piece 63 of the introducer 6 is in a first position (adjacent the proximal piece 62), the distal tip 9 of the sheath 8 is retained between the proximal and distal pieces 62, 63 of the introducer 6. In this position, as illustrated in FIG. 21C, the distal tip 9 extends around the tapered distal end 64 of the proximal piece 62, and the diameter of the distal end of the distal tip 9 is less than the dimeter of the expandable sheath 8. When the distal piece 63 of the introducer 6 is moved to the second position (spaced from the proximal piece 62) as illustrated in FIGS. 18B and 22A, the distal tip 9 of the sheath 8 is released from the introducer 6 and transitions to its biased expanded configuration.

The distal tip 9 includes a slit 92 extending longitudinally along the length of the distal tip 9 to facilitate the transition of the distal tip 9 from the expanded configuration to the unexpanded configuration. The slit 92 includes two longitudinally extending edges 93a, 93b with the spacing between the two edges increasing when the distal tip 9 transitions from the unexpanded to the expanded configuration. As provided in FIGS. 20 and 22A-22B, the slit 92 extends along a portion of the length of the distal tip 9. In other examples, the slit 92 extends along the entire length of the distal tip 9 and extends into the body portion 91 of the sheath 8. Though not shown, it is contemplated that the distal tip 9 may include additional and/or fewer slits. In some examples, the distal tip 9 may not include any slits 92.

It is contemplated that the distal tip 9 can be made from the same material as the sheath 8. The distal tip 9 can also be formed from a different material that is coupled to the distal end of the body portion 91 of the sheath 8. The distal tip 9 can be coupled to the body portion 91 by reflowing or heat bonding, using an adhesive, or any other chemical or mechanical fastener. The thickness of the distal tip 9 can be the same/correspond to the thickness of the body portion 91 of the sheath 8. In other examples, the thickness of the distal tip 9 is less than the thickness of the body portion 91 of the sheath 8. The thickness of the distal tip 9 ranges between about 0.020 inches and about 0.040 inches, preferably 0.030 inches.

As described above, the sheath hub 20 can include a sheath locking sleeve 28 for removably coupling the sheath hub 20 (and sheath 8) to the introducer locking hub 30. When the sheath hub 20 is removably coupled to the introducer locking hub 30 through the sheath locking sleeve 28, at least a portion of the introducer 6 is disposed within the central lumen of the sheath 8. The sheath locking sleeve 28 includes a sleeve body 29 having a proximal end 29a and a distal end 29b and defines a central lumen 28 extending longitudinally between the proximal end 29a and the distal end 29b. The sleeve body 29 further includes a guide 31 disposed on its outer surface 29f. The introducer locking hub 30 includes a hub body 32 that defines a locking channel 38 sized and configured to receive the guide 31 of the sleeve body 29. As described generally above, the sheath locking sleeve 28 couples to the introducer locking hub 30 via engagement between the guide 31 on the sheath locking sleeve 28 and the locking channel 38 provided in the introducer locking hub 30. The guide 31 is movable within the locking channel 38 between an unlocked position where the sheath locking sleeve 28 is rotationally and axially movable with respect to the introducer locking hub 30, and a locked position where the sheath locking sleeve 28 is axially fixed with respect to the introducer locking hub 30.

As described above, the hypotube 80 is disposed within the central lumen 61 of the introducer 6 and is coupled to the distal piece 63 of the introducer 6 to facilitate movement of the distal piece 63 with respect to the proximal piece 62. While referred to as a “hypotube” it is not meant to limit the shape to circular cross-section or particular material. Instead, the hypotube 80 defines a tubular structure sized and configured to be received within the central lumen 61 of the introducer 6. The hypotube 80 includes a body portion 81, a proximal end 81a, a distal end 81b and a central lumen 82 extending therebetween. As illustrated in FIG. 21C, the distal end 81b of the hypotube 80 is fixedly coupled within the central lumen 61 of the distal piece 63 of the introducer 6. For example, the distal end 81b of the hypotube 80 can be coupled to the distal piece 63 by at least one of a press fit, an interference fit, a snap fit, a weld, an adhesive, or any other mechanical and/or chemical fastener. The central lumen 61 of the distal piece 63, as illustrated in FIG. 21C, includes a large diameter portion 66 adjacent the proximal end of the distal piece 63 and a small diameter portion 67 adjacent the distal end of the distal piece 63. The hypotube 80 is fixedly coupled within the large diameter portion 66 of the central lumen 61 of the distal piece 63. The small diameter portion 67 is sized and configured to accommodate a guidewire for positioning the introducer 6/sheath 8 proximate the treatment site. For example, the diameter of the small diameter portion 67 and the large diameter portion 66 is greater than 0.035 inches, to accommodate a 0.035 inches diameter guidewire.

The central lumen 61 can include a tapered surface 65 extending between the proximal end of the distal piece 63 and the large diameter portion 66. When assembled, the central lumen 82 of the hypotube 80 is aligned with (and optionally coaxial with) the central lumen 40 of the introducer locking hub 30. When assembled and in the first position, the central lumen 82 of the hypotube 80 is aligned with the small diameter portion 67 of the central lumen 61 of the distal piece 63 of the introducer 6. As described above, the hypotube 80 moves (axially and rotationally) within the central lumen 61 of the proximal piece 62 of the introducer 6. Accordingly, the outer diameter of the hypotube 80 is less than the inner diameter of the proximal piece 62 of the introducer 6.

In one example, the hypotube 80 includes a metal, hard plastic (e.g., PEEK), or any combination thereof. The hypotube 80 can include surface features or texturing (e.g., laser etching or cutting, micro-machining) to increase flexibility and torque response. In other examples, the hypotube 80 includes an encapsulated or bare torque shaft (e.g., metallic torque shaft) or braided shaft (e.g., metal braiding encapsulated in a polymeric tube). The hypotube 80 can include a coating and/or liner on the outer surface and/or central lumen to increase lubricity and trackability (e.g., PTFE/FEP coatings). In some examples, the hypotube 80 includes a metal outer layer to provide stiffness and a hard-plastic inner layer to reduce the possibility of any friction between the hypotube and the guidewire, and damage to the guidewire. In some examples, different portions of the hypotube 80 are constructed from different materials to provide varying flexibility and torque response along the different lengths of the hypotube 80. For example, the proximal end 81a of the hypotube 80 can include a metal shaft while the distal end 81b of the hypotube 80 can include a braided shaft (i.e. metal braiding encapsulated in a polymeric tube) or vice versa.

In an example system, the introducer 6 includes a locking mechanism for fixing the position of the introducer 6 in at least one of the first and second positions. The locking mechanism can include a slider 70 that movably couples the hypotube 80 to the introducer locking hub 30. As explained in more detail below, the slider 70 is coupled between the introducer 6 and the hypotube 80 such that axial and/or rotational movement applied the hypotube 80 results in a corresponding movement of the distal piece 63 of the introducer 62, where movement of the distal piece 63 is independent of the proximal piece 62.

The slider 70 includes a slider body 71 having a proximal end 71a and a distal end 71b and defining a central lumen 72 extending longitudinally between the proximal and distal ends 71a,71b. The introducer 6 extends within the central lumen 72 of the slider 70, and when assembled, the slider 70 is received within the central lumen 40 of the introducer locking hub 30. The slider 70 is fixedly coupled to the hypotube 80 via connector posts 59 and includes guides/engagement features for directing movement of the hypotube 80 within the introducer locking hub 30. The connector posts 59 are fixedly coupled to the hypotube 80 and disposed within openings 71c of the slider 70. The connector posts 59 are cylindrically shaped and can be coupled to the introducer hypotube 80 by at least one of a press fit, an interference fit, a snap fit, a mechanical fastener, a weld and/or an adhesive. However, it is contemplated that the connector posts 59 may have any other regular or irregular shape that would facilitate coupling of the hypotube 80 and the slider 70. In other examples, connector posts are integrally formed onto the hypotube.

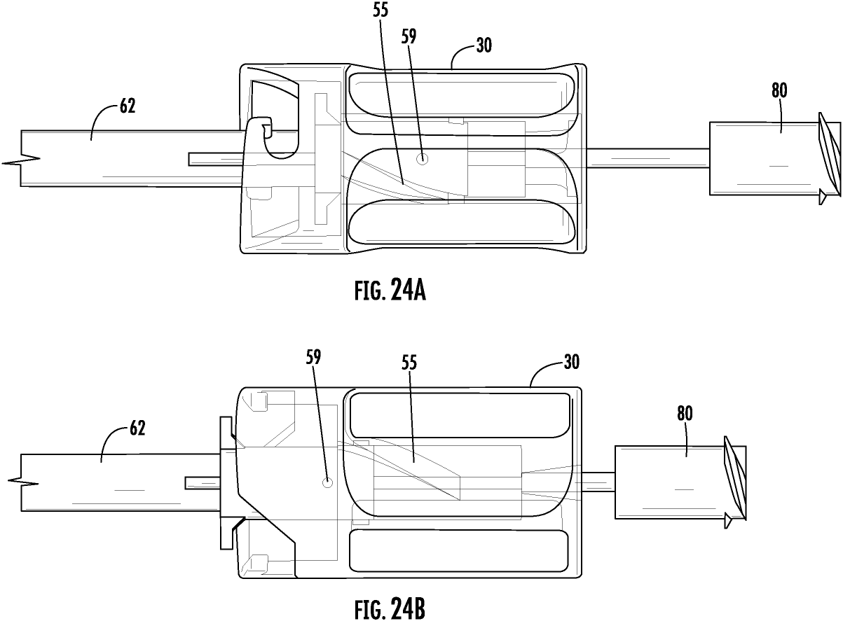

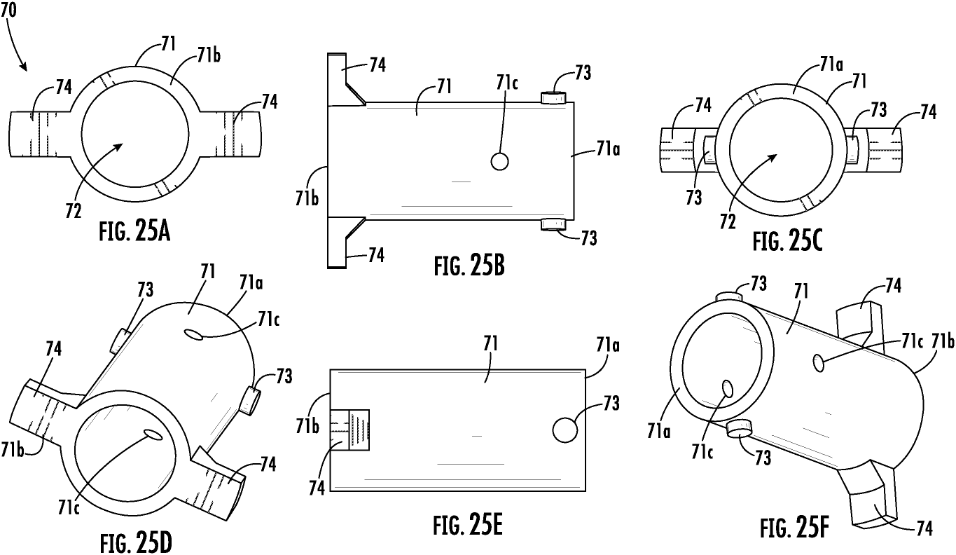

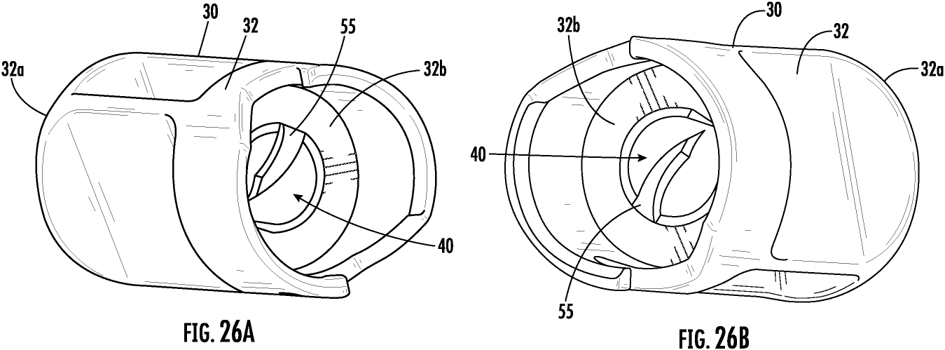

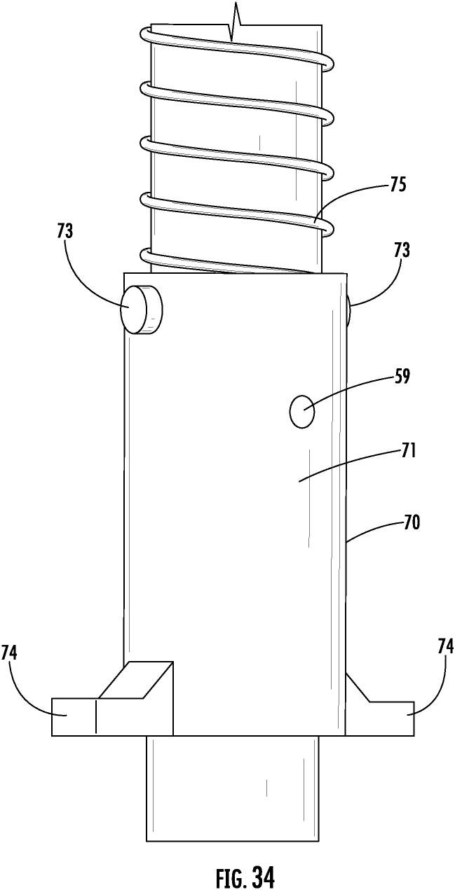

As illustrated in FIGS. 25A-25F and 34, the slider 70 includes guides 73 disposed on an outer surface of the slider body 71. The guides 73 are movable within corresponding guide channels 55 provided on the central lumen 40 of the introducer locking hub 30. Movement of the guides 73 within the guide channels 55 results in a corresponding movement of the distal piece 63 of the introducer 6 between a first position as shown in FIGS. 21A-21D, where the distal piece 63 is adjacent the proximal piece 62, and a second position as shown in FIGS. 22A-22C, where the distal piece 63 is spaced apart from the proximal piece 62. The guides 73 protrude from the outer surface of the slider body 71 and extend at least partially around a circumference of the slider body 71. As illustrated in FIGS. 25A-25F, the guides 73 comprise a cylindrically shaped projection extending from the outer surface of the slider body 71. However, it is contemplated that the guides 73 may have any other regular or irregular shape that would facilitate movement of the guides 73 within the respective guide channels 55 of the introducer locking hub 30. As provided in FIGS. 24 and 26, the guide channels 55 are formed as a recesses or grooves in a surface of the central lumen 40 of the introducer locking hub 30. The guide channels 55 extend both longitudinally along a length of the central lumen 40 and circumferentially around the central lumen 40 of the introducer locking hub 30. For example, the guide channels 55 can be described as extending helically around/along a length of the central lumen 40 of the introducer locking hub 30 or on an angle from the distal end of the introducer locking hub 30. While FIGS. 25A-25F illustrate a slider 70 with two guides 73, additional and/or fewer guides 73, and corresponding guide channels 55 are contemplated. The guides 73 are positioned equally around the slider 70. When the slider 70 includes two guides 73, the guides 73 can be positioned at circumferentially opposite locations on the slider body 71.

When assembled, the central lumen 72 of the slider 70 is aligned with the central lumen 40 of the introducer locking hub 30. For example, the central lumen 72 of the slider 70 can be coaxial with the central lumen 40 of the introducer locking hub 30.

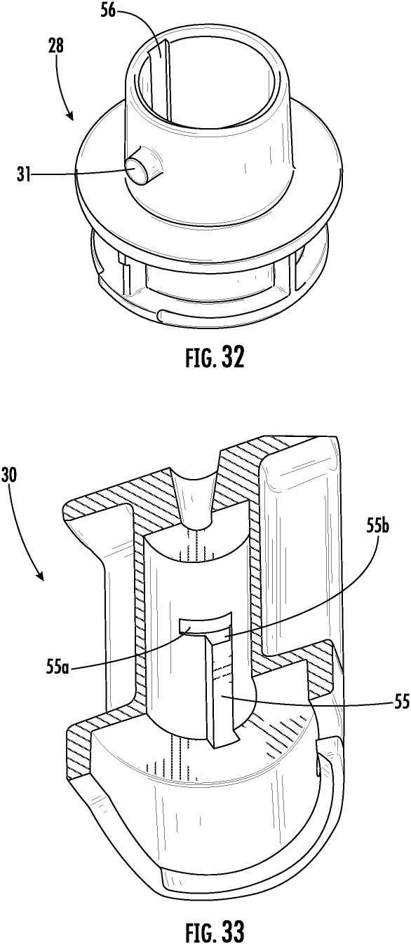

Also, the slider 70 can include restrictor projections 74 disposed on an outer surface of the slider body 71. As described above, and as illustrated in FIGS. 20, 21A, 22A and 23, the sheath 8 is coupled to a sheath hub 20. The restrictor projections 74 are movable within a corresponding restrictor channel 56 provided on the central lumen of the sheath hub 20/locking sleeve 28, shown in FIG. 32. The restrictor projections 74 protrude from the outer surface of the slider body 71 and extend at least partially around a circumference of the slider body 71. The restrictor projections 74 can comprise rectilinear shaped projection extending from the outer surface of the slider body 71. However, it is contemplated that the restrictor projections 74 may have any other regular or irregular shape (e.g., curvilinear) that would facilitate movement of the restrictor projections 74 within their corresponding restrictor channels 56. As provided in FIG. 32, the restrictor channels 56 extend longitudinally along the central lumen of the sheath hub 20/locking sleeve 28. While the figures illustrate a slider 70 with two restrictor projections 74, additional and/or fewer restrictor projections 74, and corresponding restrictor channel 56 are contemplated. The restrictor projections 74 are positioned equally around the slider 70. When the slider 70 includes two restrictor projections 74, the restrictor projections 74 can be positioned at circumferentially opposite locations on the slider body 71.

As described above, when the sheath hub 20 is coupled to the introducer locking hub 30 (e.g., via locking sleeve 28), axial movement between the expandable sheath 8 and the introducer locking hub 28 is prevented. The restrictor channels 56 extend in a longitudinal direction coplanar with a longitudinal axis 32c of the sheath hub 20/locking sleeve 28 such that rotational movement between the slider 70/hypotube 80 and the sheath hub 20/locking sleeve 28/expandable sheath 8 is prevented. Rotational movement of the introducer locking hub 28 with respect to the hypotube 80/slider 70 results in a corresponding axial movement of the guide 73 along the guide channel 55, as shown in FIGS. 24A, 24B, 26A and 26B. Accordingly, movement of the guide 73 within the guide channel 55 results in a corresponding movement of the distal piece 63 of the introducer 6 between a first position, where the distal piece is adjacent the proximal piece 62, and a second position, where the distal piece 63 is spaced apart from the proximal piece 62.

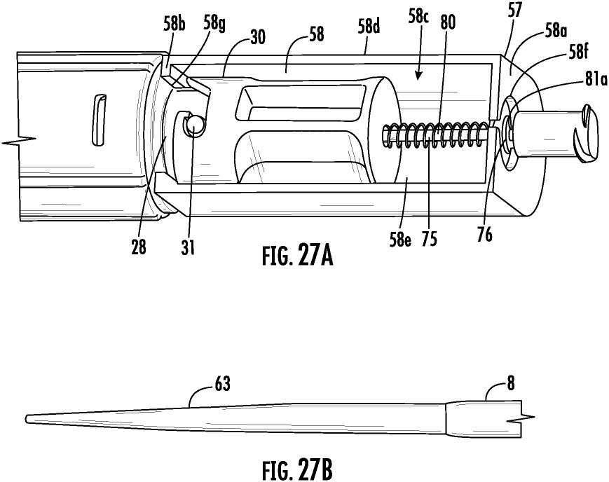

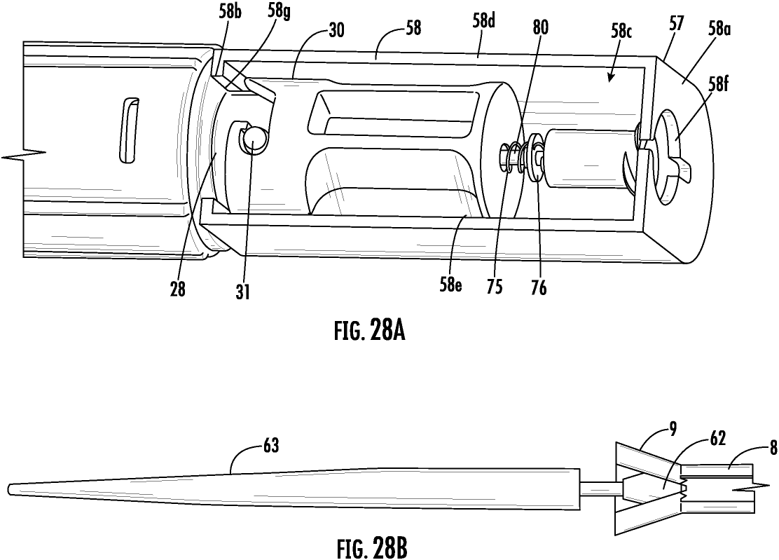

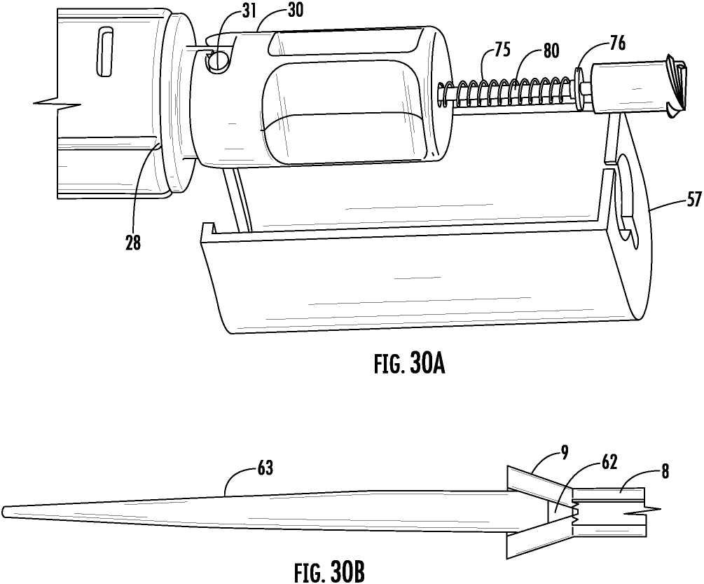

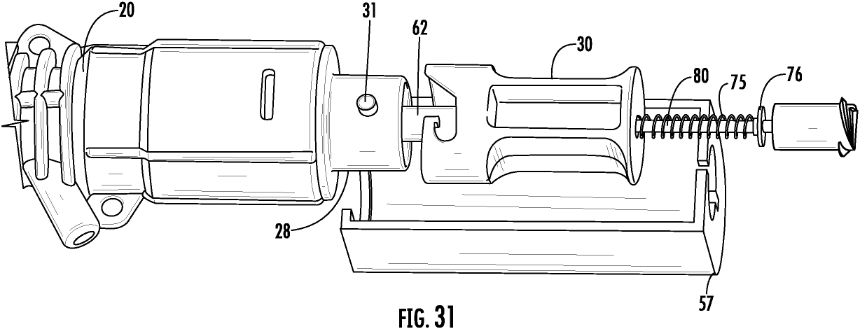

The introducer 6 may further include a spring 75 for fixing and/or biasing the position of the introducer 6 in at least one of the first and second positions. The spring 75 includes a proximal end 75a and a distal end 75b. As illustrated in FIGS. 27A-30A, the spring 75 is disposed around the body portion 81 of the hypotube 80 between a stop 76 at the proximal end 81a of the hypotube 80 and the introducer locking hub 30. In this example, the spring 75 biases the hypotube 80 in a proximal direction and the distal piece 63 of the introducer 6 in the first position.

As illustrated in FIG. 34-36C, in another example system, the spring 75 is disposed around the introducer 6 between the introducer locking hub 30 and the proximal end 71a of the slider 70. The spring 75 biases the hypotube 80 in a distal direction and the distal piece 63 of the introducer 6 in the second position. The proximal end 75a of the spring 75 abuts a radial end surface of the central lumen 40 of the introducer locking hub 30.

As described above, the locking mechanism can include a slider 70 that movably couples the hypotube 80 to the introducer locking hub 30. The slider 70 is coupled between the introducer locking hub 30 and the hypotube 80 such that axial movement applied the hypotube 80 results in a corresponding movement of the distal piece 63 of the introducer 62. The slider 70 includes a guide 73 disposed on the outer surface of the slider body 71, and the guide 73 is movable within a corresponding guide channel 55 provided on the central lumen 40 of the introducer locking hub 30. In the example systems including the spring 75, the guide channel 55 can be described as extending in a longitudinal direction coplanar with a longitudinal axis 32c of the introducer locking hub 30 such that rotational movement between the slider 70 and the introducer locking hub 30 is prevented. The movement of the guide 73 within the guide channel 55 results in a corresponding movement of the distal piece 63 of the introducer 6 between the first position and the second position.

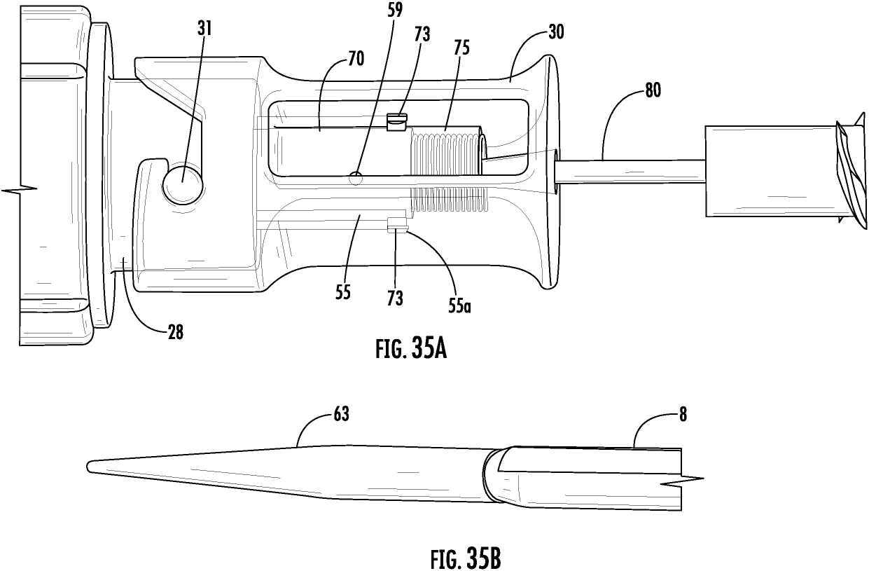

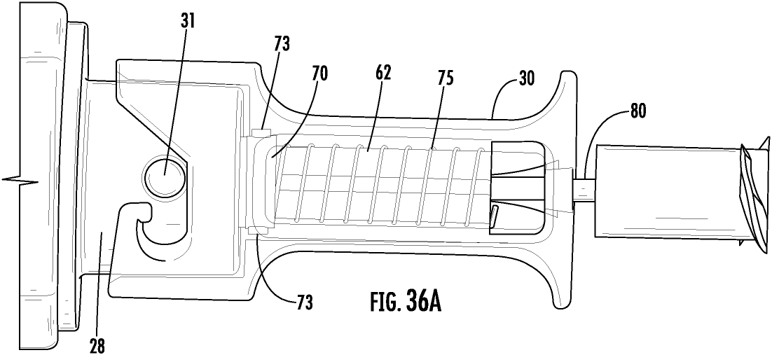

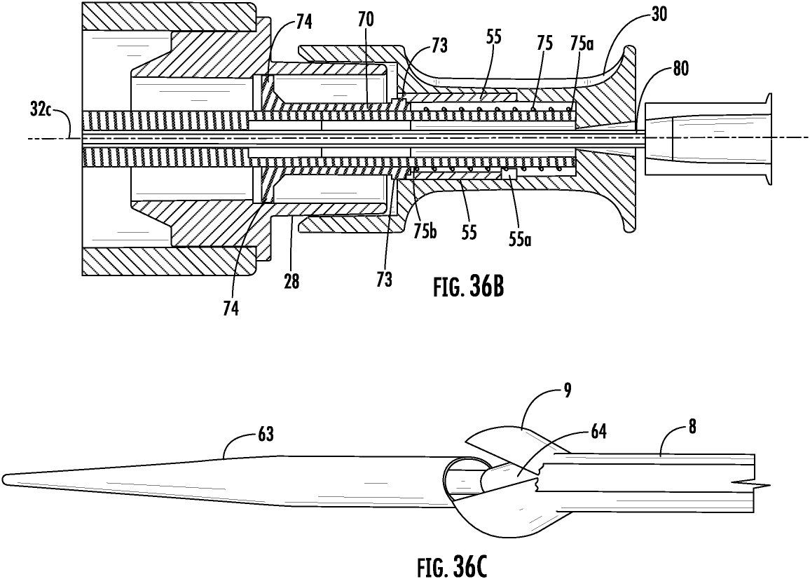

The guide channel 55 can further include a guide channel locking portion 55a for fixing the axial location of the slider 70/hypotube 80 with respect to the introducer locking hub 30/introducer 6. The locking portion 55a extends circumferentially (e.g., transverse and/or perpendicular) from the guide channel 55 and along the surface of the central lumen 40 of the introducer locking hub 30. The guide channel locking portion 55a corresponds to the guide 73 such that rotational movement of the slider 70 causes the guide 30 to engage the guide locking channel 55a. When the guide 30 is engaged with the guide locking channel 55a, the axial location of the slider 70 and the hypotube 80 is fixed with respect to the introducer locking hub 30/introducer 6. In one example, when the guide 30 is engaged with the guide locking channel 55a, the hypotube 80 and the distal piece 63 of the introducer 6 are maintained in the first position (adjacent the proximal piece 62) as provided in FIGS. 35A and 35B. Rotational movement of the hypotube 80 can cause the guide 30 to move from the locking portion 55a and into the guide channel 55, releasing the spring 75 and subsequently transitioning the distal piece 63 of the introducer into the second position as provided in FIGS. 36A-36C.

The guide channel locking portion 55a can further include a catch 55b to secure the guide 30 within the guide channel locking portion 55a and to form a partial barrier for the guide 73 within the locking portion 55a. The catch 55b can include a projection that extends from a side wall of guide channel locking portion 55a towards the center of the guide channel locking portion 55a that releasably secures the guide 73 within the guide channel locking portion 55a.

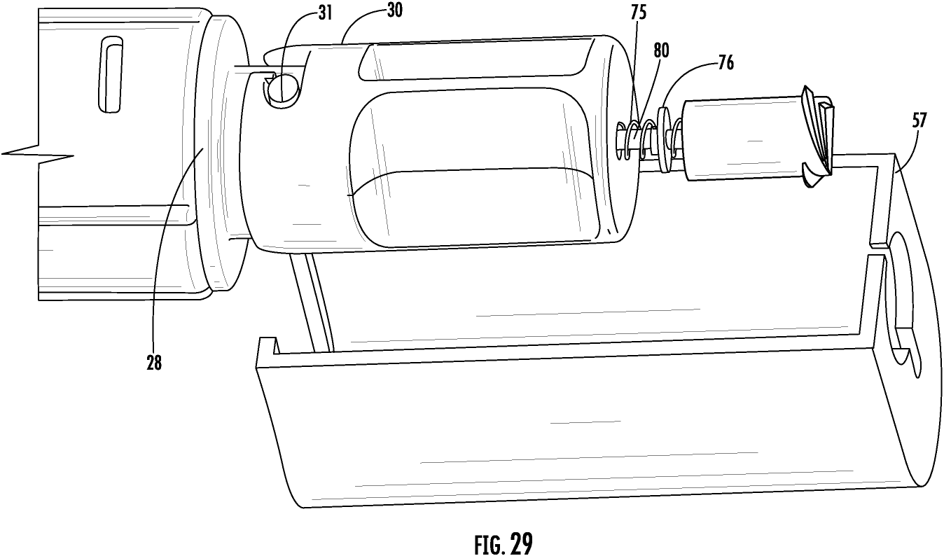

The aforementioned example systems can further include a safety lock 57 to secure the distal piece 63 of the introducer 6 in the first position and/or the second position. For example, the safety lock 57 can fix the axial position of the hypotube 80 with respect to the sheath hub 20. As illustrated in FIG. 27A-31, the safety lock 57 includes an elongated member 58 having a proximal end 58a, a distal end 58b, and a central cavity 58c therebetween as well as a first side 58d. The safety lock 57 further includes a side opening 58e in the first side 58d of the elongated member 58 for receiving at least a portion of the introducer locking hub 30 and hypotube 80 therethrough and into the central cavity 58c. The proximal end 58a of the safety lock 57 includes a proximal opening 58f for receiving the hypotube 80 and retaining the proximal end 81a of the hypotube 80. The proximal end 81a of the hypotube 80 can be retained in the safety lock 57 such that the stop 76 abuts the inner surface of the proximal end 58a adjacent to the proximal opening 58f as shown in FIG. 27A. The distal end 58b of the safety lock 57 includes a distal opening 58g for receiving at least one of the introducer locking hub 30 and the sheath hub 20. The safety lock 57 is removably couplable to the hypotube 80 and at least one of the introducer locking hub 30 and the sheath hub 20 as provided in FIG. 29.

A method of delivering a prosthetic device to a procedure device using a segmented introducer for retaining the distal end of the sheath as described above is disclosed herein. The method includes providing an introducer locking hub 30 having an elongated introducer 6 coupled to a hub body 32 of the locking hub 30. The introducer 6 includes a proximal piece 62 and a separate distal piece 63, with the proximal piece 62 being coupled to the hub body 32 of the locking hub 30 and the separate distal piece 63 being coupled to a hypotube 80. The hypotube 80 is slidably disposed within a central lumen 61 of the proximal piece 62 of the introducer 6 and the hub body 32. The introducer 6 is advanced through the central lumen of an expandable sheath 8. The sheath 8 defines an elongated tubular body 91 coupled to a sheath hub 20 and a distal tip 9 (biased to an expanded configuration) provided at a distal end of the elongated tubular body 91. As will be described below, the distal tip 9 is then retained between the proximal piece 62 and the distal piece 63 of the introducer 6. The introducer locking hub 30 is coupled to sheath hub 20 such that the axial position of the introducer locking hub 30 is fixed with respect to the sheath hub 20. Correspondingly, the axial position of the sheath 8 and the proximal piece 62 of the introducer 6 are also fixed with respect to the other. The coupled sheath 8 and introducer 6 are inserted at least partially into the vascular of the patient. The hypotube 80 is advanced within the central lumen of the proximal piece 62 of the introducer 6 such that the distal piece 63 moves from a first position adjacent the proximal piece 62 to a second position spaced away from the proximal piece 62. In the second position, the distal tip 9 of the expandable sheath 8 is released from introducer 6 and expands. The hypotube 80 is withdrawn/moved proximally within the central lumen 61 of the introducer 6 such that the distal piece 63 of the introducer 6 is brought towards (e.g., abuts) the proximal piece 62 of the introducer 6. With the proximal and distal pieces 62, 63 of the introducer 6 close together, the introducer is withdrawn from the central lumen of the sheath 8. A medical device (e.g., prosthetic heart valve) is advanced through the central lumen and the sheath 8 and delivered to a procedure site.

As described above, before the combined sheath 8 and introducer 6 are inserted into the patient vasculature, the distal tip 9 of the sheath 8 is retained between the proximal and distal pieces 62, 63 of the introducer. In this example, when the introducer locking hub 30 is initially coupled to the sheath hub 20, the hypotube 80 is advanced within the central lumen of the expandable sheath 8 such that the distal piece 63 of the introducer 6 extends through and beyond the distal end of the expandable sheath 8. The (flared/expanded) distal tip 9 of the expandable sheath 8 is compressed/moved into position against a distal end surface 64 of the proximal piece 62 of the introducer 6. The hypotube 80 is withdrawn/moved proximally within the central lumen 61 of the proximal piece 62 of the introducer 6 such that the distal piece 63 of the introducer 6 moves proximally and the distal tip 9 of the sheath 8 is retained between the proximal piece 62 and the distal piece 63. The introducer locking hub 30 is then coupled/fixed to the sheath hub 20.

In some examples, the hypotube 80 includes a locking mechanism for fixing the introducer 6 in at least one of the first and second positions. The locking mechanism includes a slider 70 fixedly coupled to the hypotube 80. The slider 70 includes a slider body 71 having a proximal end and a distal end and defining a central lumen extending longitudinally between the proximal and distal end. A guide 73 is disposed on an outer surface of the slider body 71, where the guide 73 is movable within a corresponding guide channel 55 provided on the central lumen of the introducer locking hub 30. The guide channel 55 extends helically around the central lumen of the introducer locking hub 30.

As the hypotube 80 is advanced within the central lumen of the expandable sheath 8, the introducer locking hub 30 is rotated with respect to the hypotube 80 (and/or slider 70) in a first direction to result in a corresponding axial movement of the guide 73 along the guide channel 55. Movement of the guide 73 within the guide channel 55 results in a corresponding movement of the distal piece 63 of the introducer 6 from the first position, where the distal piece is adjacent the proximal piece 62, to the second position, where the distal piece 63 is spaced apart from the proximal piece 62. When the hypotube 80 is withdrawn/moved proximally within the central lumen of the sheath 8, the introducer locking hub 30 is rotated with respect to the hypotube 80 (and/or slider 70) in a second direction to result in a corresponding axial movement of the guide 73 along the guide channel 55. This movement of the guide 73 within the guide channel 55 results in a corresponding movement of the distal piece 63 of the introducer 6 from the second position, where the distal piece 63 is adjacent the proximal piece 62, to the first position, where the distal piece 63 is spaced apart from the proximal piece 62.

In other examples, the slider 70 further includes a restrictor projection 74 that corresponds to a restrictor channel 56 defined by the sheath hub 20. The restrictor channel 56 extends longitudinally along an inner surface of the sheath hub 20. The restrictor projection 74 is axially translatable within the restrictor channel 56 such that rotational movement between the hypotube 80 (and slider 70) is prevented. When the hypotube 80 is advanced or withdrawn within the central lumen of the expandable sheath 8, the restriction projection 74 moves a corresponding length within the restrictor channel 56.

In other examples, the hypotube 80 includes a spring locking mechanism for fixing the introducer 6 in at least one of the first and second positions. The spring 75 is disposed around the hypotube 80 between a stop 76 at the proximal end of the hypotube 80 and the introducer locking hub 30. The spring 75 biases the hypotube 80 in a proximal direction and the distal piece 63 of the introducer 6 in the first position. In these examples, when the hypotube 80 is advanced within the central lumen of the expandable sheath 8, a force must be applied to the hypotube 80 in a distal direction to overcome the spring bias of the spring 75. Also, in these examples, when the hypotube 80 is withdrawn/moved proximally within the central lumen of the sheath 8 and the distal directed force on the hypotube 80 is released, the hypotube 80 is moved in a proximal direction in conjunction with the biasing effect of the spring 75.

In other examples, the spring 75 is disposed around the proximal piece 62 of the introducer 6. In these examples, the spring 75 biases the hypotube 80 in a distal direction and the distal piece 63 of the introducer 6 in the second position. When the spring 75 is released, the spring 75 applies a force to the hypotube 80 in a distal direction.

In other examples, the locking mechanism includes a slider 70 fixedly coupled to the hypotube 90. The slider 70 includes a slider body 71 having a proximal end and a distal end and defining a central lumen extending longitudinally therethrough. The slider 70 includes a guide 73 disposed on an outer surface of the slider body 71. In these examples, the guide 73 moves within a corresponding guide channel 55 provided on the central lumen of the introducer locking hub 30. The guide channel 55 extends a longitudinal direction coplanar with a longitudinal axis 32c of the introducer locking hub 30 such that rotational movement between the slider 70 and the introducer locking hub 30 is prevented. When the hypotube 80 is advanced within the central lumen of the expandable sheath 8, the hypotube 80 is moved in a distal direction such that the guide 73 advances a corresponding length within the guide channel 55.

A proximal end of the guide channel 55 includes a locking portion 55a for fixing the axial location of the slider 70/hypotube 80 with respect to the introducer locking hub 30/introducer 6. The locking portion 55a extends circumferentially around the central lumen of the introducer locking hub 30 from the proximal end of the guide channel 55. As such, rotational movement of the hypotube 80, and the slider 70 fixedly coupled thereto, causes the guide 73 to move from the guide channel 55 into the locking portion 55a. When the guide 73 engages the locking portion 55a, the hypotube 80 and the distal portion 63 of the introducer 6 are maintained in the first position, where the distal piece 63 of the introducer 6 is adjacent the proximal piece 62 of the introducer 6.

To engage the locking portion 55a, the hypotube 80 is withdrawn within the central lumen of the introducer 6 to the end of the guide channel 55. The introducer locking hub 30 is rotated with respect to the hypotube 80 (and/or slider 70) in a first direction to move the guide 73 from the guide channel 55 and into the locking portion 55a. To disengage the locking portion, the introducer locking hub 30 is rotated with respect to the hypotube 80 (and/or slide 70) in a second (opposite) direction. The guide 73 moves from the locking portion 55a to the guide channel 55. The hypotube 80 is then moved in a distal direction such that the guide 73 advances corresponding length within the guide channel 55.

A method of expanding the distal tip 9 of an expandable sheath 8 (absent insertion into the patient’s vasculature) is disclosed herein. As described above, a segmented introducer is provided for retaining the distal end of the sheath 8. The method includes providing an introducer locking hub 30 having an elongated introducer 6 coupled to a hub body 32 of the locking hub 30. The introducer 6 includes a proximal piece 62 and a separate distal piece 63, with the proximal piece 62 being coupled to the hub body 32 of the locking hub 30 and the separate distal piece 63 being coupled to a hypotube 80. The hypotube 80 is slidably disposed within a central lumen 61 of the proximal piece 62 of the introducer 6 and the hub body 32. The introducer 6 is advanced through the central lumen of an expandable sheath 8. The sheath 8 defines an elongated tubular body 91 coupled to a sheath hub 20 and a distal tip 9 (biased to an expanded configuration) provided at a distal end of the elongated tubular body 91. The distal tip 9 then moved into position such that it retained between the proximal piece 62 and the distal piece 63 of the introducer 6. For example, when the introducer locking hub 30 is initially coupled to the sheath hub 20, the hypotube 80 is advanced within the expandable sheath 8 such that the distal piece 63 of the introducer 6 extends through and beyond the distal end of the expandable sheath 8. The (flared/expanded) distal tip 9 of the sheath 8 is compressed/moved into position against a distal end surface 64 of the proximal piece 62 of the introducer 6. The hypotube 80 is then withdrawn/moved proximally within the central lumen 61 of the proximal piece 62 of the introducer 6 such that the distal piece 63 of the introducer 6 moves proximally and the distal tip 9 of the sheath 8 is retained between the proximal piece 62 and the distal piece 63. The introducer locking hub 30 is then coupled/fixed to the sheath hub 20.

The introducer locking hub 30 is then coupled to sheath hub 20 such that the axial position of the introducer locking hub 30 is fixed with respect to the sheath hub 20. Correspondingly, the axial position of the sheath 8 and the proximal piece 62 of the introducer 6 are also fixed with respect to the other.

To expand the distal tip 9 of the sheath 8, the hypotube 80 is advanced within the central lumen of the proximal piece 62 of the introducer 6 such that the distal piece 63 moves from a first position adjacent the proximal piece 62 to a second position spaced away from the proximal piece 62. In the second position, the distal tip 9 of the expandable sheath 8 is released from introducer 6 and expands. The hypotube 80 is withdrawn/moved proximally within the central lumen 61 of the introducer 6 such that the distal piece 63 of the introducer 6 is brought towards (e.g., abuts) the proximal piece 62 of the introducer 6. With the proximal and distal pieces 62, 63 of the introducer close together, the introducer is withdrawn from the central lumen of the sheath 8.

Various features of the coaxial layer structure of the sheath 8 are described in reference to FIGS. 11-14. With reference to FIG. 11, the expandable sheath 8 can include an inner layer 102 (also referred to as an inner layer), a second layer 104 disposed around and radially outward of the inner layer 102, a third layer 106 disposed around and radially outward of the second layer 104, and a fourth outer layer 108 (also referred to as an outer layer) disposed around and radially outward of the third layer 106. In the illustrated configuration, the inner layer 102 can define the lumen 112 of the sheath extending along a central axis 114.

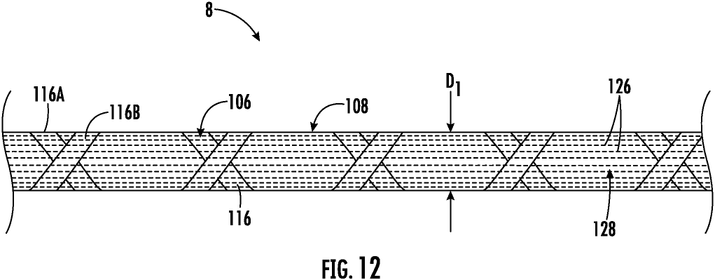

Referring to FIG. 12, when the sheath 8 is in an unexpanded state, the inner layer 102 and/or the outer layer 108 can form longitudinally-extending folds or creases such that the surface of the sheath comprises a plurality of ridges 126 (also referred to herein as “folds”). The ridges 126 can be circumferentially spaced apart from each other by longitudinally-extending valleys 128. When the sheath expands beyond its natural diameter D₁, the ridges 126 and the valleys 128 can level out or be taken up as the surface radially expands and the circumference increases, as further described below. When the sheath collapses back to its natural diameter, the ridges 126 and valleys 128 can reform.

In certain examples, the inner layer 102 and/or the outer layer 108 can comprise a relatively thin layer of polymeric material. For example, in some examples the thickness of the inner layer 102 can be from 0.01 mm to 0.5 mm, 0.02 mm to 0.4 mm, or 0.03 mm to 0.25 mm. In certain examples, the thickness of the outer layer 108 can be from 0.01 mm to 0.5 mm, 0.02 mm to 0.4 mm, or 0.03 mm to 0.25 mm.

In certain examples, the inner layer 102 and/or the outer layer 108 can comprise a lubricious, low-friction, and/or relatively non-elastic material. In particular examples, the inner layer 102 and/or the outer layer 108 can comprise a polymeric material having a modulus of elasticity of 400 MPa or greater. Exemplary materials can include ultra-high-molecular-weight polyethylene (UHMWPE) (e.g., Dyneema®), high-molecular-weight polyethylene (HMWPE), or polyether ether ketone (PEEK). With regard to the inner layer 102 in particular, such low coefficient of friction materials can facilitate passage of the prosthetic device through the lumen 112. Other suitable materials for the inner and outer layers can include polytetrafluoroethylene (PTFE), expanded polytetrafluoroethylene (ePTFE), ethylene tetrafluoroethylene (ETFE), nylon, polyethylene, polyether block amide (e.g., Pebax), and/or combinations of any of the above. Some examples of a sheath 8 can include a lubricious liner on the inner surface of the inner layer 102. Examples of suitable lubricious liners include materials that can further reduce the coefficient of friction of the inner layer 102, such as PTFE, polyethylene, polyvinylidene fluoride, and combinations thereof. Suitable materials for a lubricious liner also include other materials desirably having a coefficient of friction of 0.1 or less.

Additionally, some examples of the sheath 8 can include an exterior hydrophilic coating on the outer surface of the outer layer 108. Such a hydrophilic coating can facilitate insertion of the sheath 8 into a patient’s vessel, reducing potential damage. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N.V, Heerlen, the Netherlands), as well as other hydrophilic coatings (e.g., PTFE, polyethylene, polyvinylidene fluoride), are also suitable for use with the sheath 8. Such hydrophilic coatings may also be included on the inner surface of the inner layer 102 to reduce friction between the sheath and the delivery system, thereby facilitating use and improving safety. In some examples, a hydrophobic coating, such as Perylene, may be used on the outer surface of the outer layer 108 or the inner surface of the inner layer 102 in order to reduce friction.

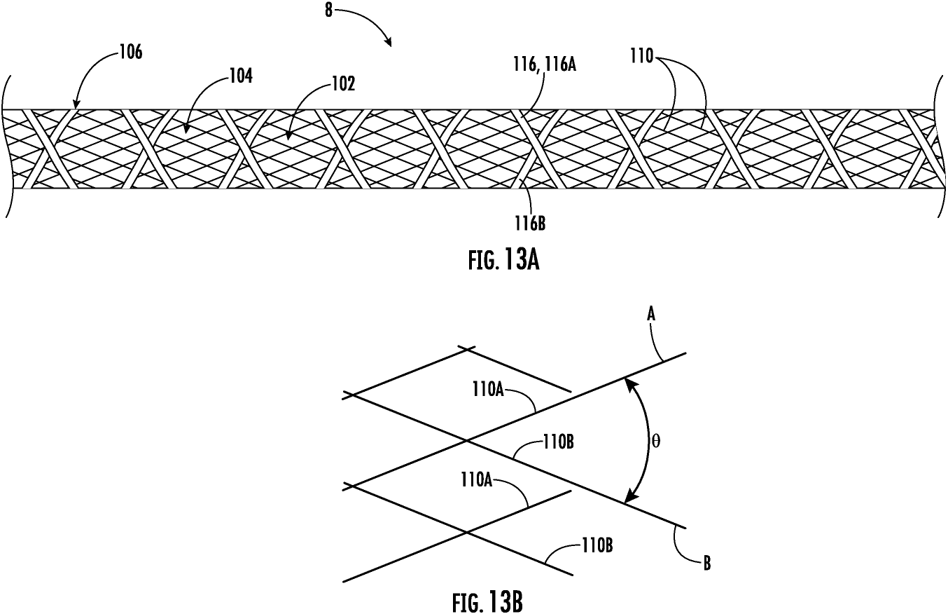

In certain examples, the second layer 104 can be a braided layer. FIGS. 13A and 13B illustrate the sheath 8 with the outer layer 108 removed to expose the elastic third layer 106. With reference to FIGS. 13A and 13B, the braided second layer 104 can comprise a plurality of members or filaments 110 (e.g., metallic or synthetic wires or fibers) braided together. The braided second layer 104 can have any desired number of filaments 110, which can be oriented and braided together along any suitable number of axes. For example, with reference to FIG. 13B, the filaments 110 can include a first set of filaments 110A oriented parallel to a first axis A, and a second set of filaments 110B oriented parallel to a second axis B. The filaments 110A and 110B can be braided together in a biaxial braid such that filaments 110A oriented along axis A form an angle θ with the filaments 110B oriented along axis B. In certain examples, the angle θ can be from 5° to 70°, 10° to 60°, 10° to 50°, or 10° to 45°. In the illustrated example, the angle θ is 45°. In other examples, the filaments 110 can also be oriented along three axes and braided in a triaxial braid, or oriented along any number of axes and braided in any suitable braid pattern. The braided second layer 104 can extend along substantially the entire length L of the sheath 8, or alternatively, can extend only along a portion of the length of the sheath. In particular examples, the filaments 110 can be wires made from metal (e.g., Nitinol, stainless steel, etc.), or any of various polymers or polymer composite materials, such as carbon fiber. In certain examples, the filaments 110 can be round, and can have a diameter of from 0.01 mm to 0.5 mm, 0.03 mm to 0.4 mm, or 0.05 mm to 0.25 mm. In other examples, the filaments 110 can have a flat cross-section with dimensions of 0.01 mm x 0.01 mm to 0.5 mm x 0.5 mm, or 0.05 mm x 0.05 mm to 0.25 mm x 0.25 mm. In one example, filaments 110 having a flat cross-section can have dimensions of 0.1 mm x 0.2 mm. However, other geometries and sizes are also suitable for certain examples. If braided wire is used, the braid density can be varied. Some examples have a braid density of from ten picks per inch to eighty picks per inch, and can include eight wires, sixteen wires, or up to fifty-two wires in various braid patterns. In other examples, the second layer 104 can be laser cut from a tube, or laser-cut, stamped, punched, etc., from sheet stock and rolled into a tubular configuration. The second layer 104 can also be woven or knitted, as desired.

The third layer 106 can be a resilient, elastic layer (also referred to as an elastic material layer). In certain examples, the elastic third layer 106 can be configured to apply force to the inner layer 102 and second layer 104 in a radial direction (e.g., toward the central axis 114 of the sheath) when the sheath expands beyond its natural diameter by passage of the delivery apparatus through the sheath. Stated differently, the elastic third layer 106 can be configured to apply encircling pressure to the layers of the sheath beneath the elastic third layer 106 to counteract expansion of the sheath. The radially inwardly directed force is sufficient to cause the sheath to collapse radially back to its unexpanded state after the delivery apparatus is passed through the sheath.

In the illustrated example, the elastic third layer 106 can comprise one or more members configured as strands, ribbons, or bands 116 helically wrapped around the braided second layer 104. For example, in the illustrated example the elastic third layer 106 comprises two elastic bands 116A and 116B wrapped around the braided second layer 104 with opposite helicity, although the elastic layer may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some examples, the elastic layer can comprise an elastomeric material having a modulus of elasticity of 200 MPa or less. In some examples, the elastic third layer 106 can comprise a material exhibiting an elongation to break of 200% or greater, or an elongation to break of 400% or greater. The elastic third layer 106 can also take other forms, such as a tubular layer comprising an elastomeric material, a mesh, a shrinkable polymer layer such as a heat-shrink tubing layer, etc. In lieu of, or in addition to, the elastic third layer 106, the sheath 8 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In other examples, the elastic third layer 106 can also be radially outward of the polymeric outer layer 108.

In certain examples, one or both of the inner layer 102 and/or the outer layer 108 can be configured to resist axial elongation of the sheath 8 when the sheath expands. More particularly, one or both of the inner layer 102 and/or the outer layer 108 can resist stretching against longitudinal forces caused by friction between a prosthetic device and the inner surface of the sheath such that the length L remains substantially constant as the sheath expands and contracts. As used herein with reference to the length L of the sheath, the term “substantially constant” means that the length L of the sheath increases by not more than 1%, by not more than 5%, by not more than 10%, by not more than 15%, or by not more than 20%. Meanwhile, with reference to FIG. 13B, the filaments 110A and 110B of the braided second layer 104 can be allowed to move angularly relative to each other such that the angle θ changes as the sheath expands and contracts. This, in combination with the longitudinal ridges 126 (or folds) in the inner layer 102 and outer layer 108, can allow the lumen 112 of the sheath to expand as a prosthetic device is advanced through it.

For example, in some examples the inner layer 102 and the outer layer 108 can be heat-bonded during the manufacturing process such that the braided second layer 104 and the elastic third layer 106 are encapsulated between the inner layer 102 and outer layer 108. More specifically, in certain examples the inner layer 102 and the outer layer 108 can be adhered to each other through the spaces between the filaments 110 of the braided second layer 104 and/or the spaces between the elastic bands 116. The inner layer 102 and outer layer 108 can also be bonded or adhered together at the proximal and/or distal ends of the sheath. In certain examples, the inner layer 102 and outer layer 108 are not adhered to the filaments 110. This can allow the filaments 110 to move angularly relative to each other, and relative to the inner layer 102 and outer layer 108, allowing the diameter of the braided second layer 104, and thereby the diameter of the sheath, to increase or decrease. As the angle θ between the filaments 110A and 110B changes, the length of the braided second layer 104 can also change. For example, as the angle θ increases, the braided second layer 104 can foreshorten, and as the angle θ decreases, the braided second layer 104 can lengthen to the extent permitted by the areas where the inner layer 102 and outer layer 108 are bonded. However, because the braided second layer 104 is not adhered to the inner layer 102 and outer layer 108, the change in length of the braided layer that accompanies a change in the angle θ between the filaments 110A and 110B does not result in a significant change in the length L of the sheath.

FIG. 13 illustrates radial expansion of the sheath 8 as a prosthetic device (e.g., implant 12) is passed through the sheath in the direction of arrow 132 (e.g., distally). As the prosthetic device (implant 12) is advanced through the sheath 8, the sheath can resiliently expand to a second diameter D₂ that corresponds to a size or diameter of the prosthetic device. As the prosthetic device (implant 12) is advanced through the sheath 8, the prosthetic device can apply longitudinal force to the sheath in the direction of motion by virtue of the frictional contact between the prosthetic device and the inner surface of the sheath. However, as noted above, the inner layer 102 and/or the outer layer 108 can resist axial elongation such that the length L of the sheath remains constant, or substantially constant. This can reduce or prevent the braided layer second layer 104 from lengthening, and thereby constricting the lumen 112.

Meanwhile, the angle θ between the filaments 110A and 110B can increase as the sheath expands to the second diameter D₂ to accommodate the prosthetic valve. This can cause the braided second layer 104 to foreshorten. However, because the filaments 110 are not engaged or adhered to the inner layer 102 or outer layer 108, the shortening of the braided second layer 104 attendant to an increase in the angle θ does not affect the overall length L of the sheath. Moreover, because of the longitudinally-extending ridges 126 (or folds) formed in the inner layer 102 and outer layer 108, the inner layer 102 and outer layer 108 can expand to the second diameter D₂ without rupturing, in spite of being relatively thin and relatively non-elastic. In this manner, the sheath 8 can resiliently expand from its natural diameter D₁ to a second diameter D₂ that is larger than the diameter D₁ as a prosthetic device is advanced through the sheath, without lengthening, and without constricting. Thus, the force required to push the prosthetic implant through the sheath is significantly reduced.

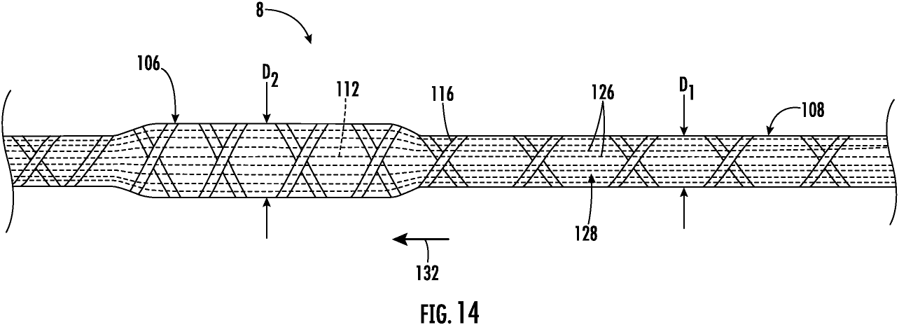

Additionally, because of the radial force applied by the elastic third layer 106, the radial expansion of the sheath 8 can be localized to the specific portion of the sheath occupied by the prosthetic device. For example, with reference to FIG. 14, as the prosthetic device (implant 12) moves distally through the sheath 8, the portion of the sheath immediately proximal to the prosthetic device (e.g., implant 12) can radially collapse back to the initial diameter D₁ under the influence of the elastic third layer 106. The inner layer 102 and outer layer 108 can also buckle as the circumference of the sheath is reduced, causing the ridges 126 and the valleys 128 to reform. This can reduce the size of the sheath required to introduce a prosthetic device of a given size. Additionally, the temporary, localized nature of the expansion can reduce trauma to the blood vessel into which the sheath is inserted, along with the surrounding tissue, because only the portion of the sheath occupied by the prosthetic device expands beyond the sheath’s natural diameter and the sheath collapses back to the initial diameter once the device has passed. This limits the amount of tissue that must be stretched in order to introduce the prosthetic device, and the amount of time for which a given portion of the vessel must be dilated.

A method for controlling articulation/bending of a delivery sheath is disclosed herein. The method includes providing an expandable introducer sheath with a central lumen extending therethrough and a pull wire coupled to the distal end of the sheath. A tension force is applied to a proximal end of the pull wire resulting in a corresponding bending motion/curvature of the sheath in a direction away from the longitudinal axis of the sheath. When the force is released from the pull wire the sheath returns back toward the longitudinal axis of the sheath. In some sheaths the distal tip portion includes a feature (e.g., is constructed from a more flexible material, includes a surface treatment, includes slots/grooves on the outer surface of the sheath) that facilitate curving of the distal tip portion with respect to the remainder of the sheath. In these examples, only the distal tip portion of the sheath curves in response to tension being applied on the pull wire. In another example, the sheath curves along an entire length of the sheath.

Another example method for controlling articulation/bending of a delivery sheath includes providing an expandable introducer sheath with a central lumen extending therethrough, where a distal tip portion of the sheath is more flexible than a proximal portion of the sheath. A stylet is inserted into the central lumen of the sheath, the stylet including a curved portion for effecting a curve on the sheath. When the curved portion of the stylet is aligned with the distal tip portion, the distal tip portion in a direction corresponding to the curvature of the stylet. For example, the stylet may include a curved portion having a curvature extending in a direction away from the longitudinal axis of the sheath. When the curved portion of the stylet aligns with the more flexible portion of the sheath, a corresponding curving effect is accomplished. In some examples, the stylet includes a curved portion that curves in a direction away from the longitudinal axis of the sheath, resulting in a corresponding curvature in the sheath. Removing the stylet, at least partially, from the central lumen of the sheath, such that the curved portion of the stylet is no longer aligned with more flexible portion of the sheath will result in the sheath returning to its original curvature.

A method of delivering a medical device using an articulating introducer sheath is disclosed herein. The method comprises inserting a sheath at least partially into the blood vessel of the patient. In an example method, the sheath is introduced into the patient via the femoral vein. The distal end of the sheath is then advanced to a first location proximate the treatment site. For example, in a transseptal approach for mitral valve repair/replacement, the sheath is advanced via the inferior vena cava into the right atrium. In some examples, a guidewire is positioned at the treatment site and the sheath is advanced over the guidewire.

The distal end of the sheath is curved to facilitate access to the treatment site. In the example of a transseptal approach for mitral valve replacement, it is necessary to curve the end of the sheath to access the mitral valve through the foramen ovalis.

With the end of the sheath curved, the sheath and/or a medical device can be advanced from the first location (within the right atrium) to the treatment site (at the mitral valve). Accessing the treatment site may require creating an opening the heart tissue (e.g., foramen ovalis) of the patient. In this example, a cutting instrument can be advanced through the sheath to create an opening in the patient’s heart tissue. Cutting instruments include, for example, a Brockenbrough-type needle. Using the cutting instrument, an incision is made in the heart tissue (e.g., foramen ovalis), the cutting instrument withdrawn, and the distal end of the sheath is advanced through the opening in the patient’s heart tissue.

A medical device (e.g., an implant) is advanced through the central lumen of the sheath to the treatment site. Where the distal end of the sheath is provided through an opening in the patient’s heart tissue, advancing the medical device to the treatment site includes advancing the medical device, via the sheath, through the opening in the heart tissue.

The sheath is locally expanding from an initial condition/diameter to a locally expanded condition/diameter by the outwardly directed radial force of the medical device against the inner wall of the central lumen of the sheath. The sheath is then locally contracted from the locally expanded condition at least partially back to the initial condition using inwardly directed radial force an elastic feature of the sheath.

With the distal end of the sheath positioned at the treatment site, the medical device is deployed beyond the sheath and delivered to the patient. When the distal end of the sheath provided through an opening in the patient’s heart tissue, opening within the heart tissue is expanded by the passing medical device and released back towards its initial condition upon passing of the implant. Where typical transseptal approach procedures require large incision size and the use of a shunt or access tube to maintain the opening in foramen ovalis, the locally expanding, articulating sheath of the present disclosure allows for a much smaller incision opening as only the sheath needs to be inserted (and maintained) in the opening in the foramen ovalis. As a result, the incision size is reduced from approximately 1.5” to less than 0.5”. Moreover, because the incision is only expanded temporarily during passage of the implant, and a large opening does not have to be maintained (e.g., by shunt), less stress is placed on tissue surrounding the opening. There is also less concern for adverse events post procedure related to closing the incision or leakage around a shunt left in place because the incision is significantly smaller, and shuts are not required.

### EXEMPLARY ASPECTS

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: An introducer sheath system comprising: an introducer locking hub comprising a hub body having a proximal end and a distal end and defining a central lumen extending longitudinally between the proximal end and the distal end; an introducer coupled to the introducer locking hub, the introducer extending beyond the distal end of the hub body and having a central lumen extending therethrough, the introducer defining an elongated body comprising a proximal piece and a separate distal piece, the proximal piece coupled to the introducer locking hub; and a hypotube disposed within the central lumen of the introducer, the hypotube having a proximal end and a distal end and defining a central lumen extending longitudinally between the proximal end and the distal end, wherein the distal end of the hypotube is coupled to the distal piece of the introducer and at least a portion of the hypotube is slidably disposed in the central lumen of the proximal piece of the introducer such that movement of the hypotube within the central lumen of the proximal piece causes the distal piece of the introducer to move with respect to the proximal piece of the introducer.

Example 2: The introducer sheath system according to any example herein, particularly example 1, wherein the distal piece of the introducer is movable from a first position adjacent the proximal piece to a second position spaced from the proximal piece.

Example 3: The introducer sheath system according to any example herein, particularly example 2, wherein axial movement of the hypotube within the central lumen of the proximal piece of the introducer causes a corresponding axial movement of the distal piece of the introducer, wherein, when the introducer is in the first position, axial movement of the hypotube in a distal direction causes the distal piece to move from the first position to the second position, wherein, when the introducer is in the second position, axial movement of the hypotube in a proximal direction causes the distal piece to move from the second position to the first position.

Example 4: The introducer sheath system according to any example herein, particularly example 3, wherein the spacing between the distal piece and the proximal piece in the second position ranges between about 0.25 inches and about 1.25 inches, between about 0.5 inches and about 1.0 inches, where the spacing is measured along an outer diameter of the introducer.

Example 5: The introducer sheath system according to any example herein, particularly examples 1-4, wherein rotational movement of the hypotube within the central lumen of the proximal piece of the introducer causes a corresponding rotational movement of the distal piece of the introducer.

Example 6: The introducer sheath system according to any example herein, particularly examples 1-5, wherein the central lumen of the introducer is aligned with the central lumen of the introducer locking hub.

Example 7: The introducer sheath system according to any example herein, particularly examples 1-6, wherein the central lumen of the introducer is coaxial with the central lumen of the introducer locking hub.

Example 8: The introducer sheath system according to any example herein, particularly examples 1-7, wherein the proximal piece of the introducer is fixedly coupled to the introducer locking hub.

Example 9: The introducer sheath system according to any example herein, particularly examples 1-8, wherein the proximal piece of the introducer is coupled to the introducer locking hub by at least one of a press fit, an interference fit, a snap fit, a mechanical fastener, a weld and an adhesive.

Example 10: The introducer sheath system according to any example herein, particularly examples 2-9, wherein the proximal piece of the introducer includes a tapered distal end, wherein at least a portion of the tapered distal end of the proximal piece is received within the central lumen of the distal piece when the introducer is in the first position.

Example 11: The introducer sheath system according to any example herein, particularly example 10, wherein the central lumen of the distal piece includes a corresponding tapered surface.

Example 12: The introducer sheath system according to any example herein, particularly examples 1-11, wherein an outer diameter of the proximal piece is the same as an outer diameter of the distal piece.

Example 13: The introducer sheath system according to any example herein, particularly examples 1-12, wherein the outer diameter of the proximal and distal pieces ranges between about 0.131 inches (10F) and about 0.236 inches (18F), preferably about 0.170 inches (14F), wherein the central lumen has a diameter greater than 0.035 inches.

Example 14: The introducer sheath system according to any example herein, particularly examples 1-11, wherein an outer diameter of the proximal piece is less than an outer diameter of the distal piece.

Example 15: The introducer sheath system according to any example herein, particularly examples 1-14, wherein the distal piece includes a tapered distal nosecone.

Example 16: The introducer sheath system according to any example herein, particularly examples 1-15, further including an expandable sheath coupled to a sheath hub, the sheath defining an elongated tubular body with a central lumen extending therethrough, the central lumen of the sheath aligned with the central lumens of the sheath hub and the introducer locking hub.

Example 17: The introducer sheath system according to any example herein, particularly examples 1-16, wherein the introducer locking hub is removably coupled to the sheath hub and at least a portion of the introducer is disposed within the central lumen of the sheath member.

Example 18: The introducer sheath system according to any example herein, particularly examples 1-17, wherein the sheath includes an expandable body portion and distal tip portion provided at a distal end of the body portion, wherein the distal tip portion is biased in an expanded configuration.

Example 19: The introducer sheath system according to any example herein, particularly example 18, wherein, when the distal piece of the introducer is in a first position adjacent the proximal piece, the distal tip portion of the sheath is retained between the proximal and distal pieces of the introducer, wherein, when the distal piece of the introducer is in a second position spaced from the proximal piece, the distal tip portion of the sheath is released from the introducer and transitions to the expanded configuration.

Example 20: The introducer sheath system according to any example herein, particularly examples 18-19, wherein the distal tip portion is heat set in the expanded configuration.

Example 21: The introducer sheath system according to any example herein, particularly examples 18-20, wherein the diameter of a distal end of the distal tip portion in the expanded configuration is greater than a diameter of the expandable sheath, wherein an outer diameter of the body portion of the sheath in an unexpanded configuration ranges between about 0.300 inches and about 0.200 inches.

Example 22: The introducer sheath system according to any example herein, particularly examples 18-21, wherein the distal tip portion defines a frustoconical shape when in the expanded configuration.

Example 23: The introducer sheath system according to any example herein, particularly examples 18-22, wherein the distal tip portion includes a slit extending longitudinally along a length of the distal tip portion.

Example 24: The introducer sheath system according to any example herein, particularly example 23, wherein the slit extends along a portion of an overall length of the distal tip portion.

Example 25: The introducer sheath system according to any example herein, particularly example 23, wherein the slit extends beyond a proximal end of the distal tip portion and along a length of the expandable sheath.

Example 26: The introducer sheath system according to any example herein, particularly examples 23-25, wherein spacing between longitudinal edges of the slit increases when the distal tip portion transitions from an unexpanded to the expanded configuration.

Example 27: The introducer sheath system according to any example herein, particularly examples 18-26, wherein at least one of the body portion of the expandable sheath and the distal tip portion comprise a shape memory material.

Example 28: The introducer sheath system according to any example herein, particularly examples 18-26, wherein at least one of the body portion of the expandable sheath and the distal tip portion comprise nitinol.

Example 29: The introducer sheath system according to any example herein, particularly examples 18-28, wherein a thickness of the distal tip portion corresponds to a thickness of the body portion of the sheath.

Example 30: The introducer sheath system according to any example herein, particularly example 29, wherein the thickness of the distal tip portion ranges between about 0.020 inches and about 0.040 inches, preferably about 0.030 inches.

Example 31: The introducer sheath system according to any example herein, particularly examples 18-30, wherein a thickness of the distal tip portion is less than a thickness of the body portion of the sheath.

Example 32: The introducer sheath system according to any example herein, particularly examples 18-31, wherein the sheath hub includes a sheath locking sleeve for removably coupling the sheath hub to the introducer locking hub.

Example 33: The introducer sheath system according to any example herein, particularly example 32, wherein the sheath locking sleeve comprises a sleeve body having a proximal end and a distal end and defining a central lumen extending longitudinally between the proximal end and the distal end, a guide disposed on an outer surface of the sleeve body, wherein the hub body of the introducer locking hub further defines a locking channel disposed on the hub body, wherein the guide is movable within the locking channel between an unlocked position where the sheath locking sleeve is rotationally and axially movable with respect to the introducer locking hub, and a locked position where the sheath locking sleeve is axially fixed with respect to the introducer locking hub.

Example 34: The introducer sheath system according to any example herein, particularly examples 1-33, wherein the central lumen of the hypotube is aligned with the central lumen of the introducer locking hub.

Example 35: The introducer sheath system according to any example herein, particularly examples 1-34, wherein the central lumen of the hypotube is coaxial with the central lumen of the introducer locking hub.

Example 36: The introducer sheath system according to any example herein, particularly examples 1-35, wherein a distal end of the hypotube is fixedly coupled within the central lumen of the distal piece of the introducer.

Example 37: The introducer sheath system according to any example herein, particularly examples 1-36, wherein a distal end of the hypotube is coupled to the central lumen of the distal piece by at least one of a press fit, an interference fit, a snap fit, a mechanical fastener, a weld and an adhesive.

Example 38: The introducer sheath system according to any example herein, particularly examples 1-37, wherein the central lumen of the distal piece includes a large diameter portion adjacent a proximal end of the distal piece, and a small diameter portion adjacent a distal end of the distal piece, wherein the hypotube is fixedly coupled within the large diameter portion of the central lumen of the distal piece.

Example 39: The introducer sheath system according to any example herein, particularly example 38, wherein the central lumen of the distal piece includes a decreasing tapered surface extending between the proximal end of the distal piece and the large diameter portion.

Example 40: The introducer sheath system according to any example herein, particularly examples 38-39, wherein the central lumen of the hypotube is aligned with the small diameter portion of the central lumen of the distal piece of the introducer.

Example 41: The introducer sheath system according to any example herein, particularly examples 1-40, wherein the central lumen of the hypotube has a diameter greater than 0.035 inches.

Example 42: The introducer sheath system according to any example herein, particularly examples 2-41, including a locking mechanism for fixing the introducer in at least one of the first and second positions.

Example 43: The introducer sheath system according to any example herein, particularly example 42, wherein the locking mechanism includes a slider fixedly coupled to the hypotube, the slider comprising a slider body having a proximal end and a distal end and defining a central lumen extending longitudinally between the proximal and distal end, wherein the slider movably couples the hypotube to the introducer locking hub.

Example 44: The introducer sheath system according to any example herein, particularly example 43, wherein the slider includes a guide disposed on an outer surface of the slider body, wherein the guide is movable within a corresponding guide channel provided on the central lumen of the introducer locking hub, wherein movement of the guide within the guide channel results in a corresponding movement of the distal piece of the introducer between a first position, where the distal piece is adjacent the proximal piece, and a second position, where the distal piece is spaced apart from the proximal piece.

Example 45: The introducer sheath system according to any example herein, particularly example 44, wherein the guide protrudes from the outer surface of the slider body and extends at least partially around a circumference of the slider body.

Example 46: The introducer sheath system according to any example herein, particularly examples 44-45, wherein the guide comprises a cylindrical shaped projection extending from the outer surface of the slider body.

Example 47: The introducer sheath system according to any example herein, particularly examples 44-46, wherein the guide channel extends helically around the central lumen of the introducer locking hub.

Example 48: The introducer sheath system according to any example herein, particularly examples 44-47, wherein the guide channel is formed as a recess or groove in a surface of the central lumen of the introducer locking hub.

Example 49: The introducer sheath system according to any example herein, particularly examples 44-48, wherein the guide channel extends along a length of the central lumen of the introducer locking hub.

Example 50: The introducer sheath system according to any example herein, particularly examples 44-47, wherein the slider includes a second guide disposed on the outer surface of the slider body, the second guide circumferentially opposite the guide, wherein the second guide is movable within a corresponding second guide channel provided on the central lumen of the introducer locking hub, wherein movement of the second guide within the second guide channel results in a corresponding movement of the distal piece of the introducer between the first position and the second position.

Example 51: The introducer sheath system according to any example herein, particularly examples 44-50, wherein the central lumen of the slider is aligned with the central lumen of the introducer locking hub.

Example 52: The introducer sheath system according to any example herein, particularly examples 44-51, wherein the central lumen of the slider is coaxial with the central lumen of the introducer locking hub.

Example 53: The introducer sheath system according to any example herein, particularly examples 43-52, further including an expandable sheath coupled to a sheath hub, the sheath defining an elongated tubular body with a central lumen extending therethrough, the central lumen of the sheath aligned with the central lumens of the sheath hub and the introducer locking hub, wherein the slider includes a restrictor projection disposed on an outer surface of the slider body, wherein the restrictor projection is movable within a corresponding restrictor channel provided on the central lumen of the sheath hub.

Example 54: The introducer sheath system according to any example herein, particularly example 53, wherein the sheath hub includes a locking sleeve for removably coupling the sheath hub to the introducer locking hub, wherein the sheath locking sleeve comprises a sleeve body having a proximal end and a distal end and defining a central lumen extending longitudinally between the proximal end and the distal end, a guide disposed on an outer surface of the sleeve body, wherein the restrictor channel is provided on the central lumen of the locking sleeve.

Example 55: The introducer sheath system according to any example herein, particularly examples 53-54, wherein the restrictor projection protrudes from the outer surface of the slider body and extends at least partially around a circumference of the slider body.

Example 56: The introducer sheath system according to any example herein, particularly examples 53-55, wherein the restrictor projection comprises a curvilinear or rectilinear shaped projection extending from the outer surface of the slider body.

Example 57: The introducer sheath system according to any example herein, particularly examples 53-56, wherein the restrictor channel extends longitudinally along the central lumen of the sheath hub.

Example 58: The introducer sheath system according to any example herein, particularly examples 53-56, wherein, when the sheath hub is coupled to the introducer locking hub, axial movement between the expandable sheath and the introducer locking hub is prevented, wherein the restrictor channel extends in a longitudinal direction coplanar with a longitudinal axis of the sheath hub such that rotational movement between the slider/hypotube and the sheath hub/expandable sheath is prevented, wherein rotational movement of the introducer locking hub with respect to the hypotube/slider results in a corresponding axial movement of the guide along the guide channel, where movement of the guide within the guide channel results in a corresponding movement of the distal piece of the introducer between a first position, where the distal piece is adjacent the proximal piece, and a second position, where the distal piece is spaced apart from the proximal piece.

Example 59: The introducer sheath system according to any example herein, particularly examples 43-58, wherein the locking mechanism includes a spring disposed around the hypotube between a stop at the proximal end of the hypotube and the introducer locking hub, wherein the spring biases the hypotube in a proximal direction and the distal piece of the introducer in the first position.

Example 60: The introducer sheath system according to any example herein, particularly examples 43-59, wherein the locking mechanism includes a spring disposed around the introducer between the introducer locking hub and the proximal end of the slider, wherein the spring biases the hypotube in a distal direction and the distal piece of the introducer in the second position.

Example 61: The introducer sheath system according to any example herein, particularly example 60, wherein a proximal end of the spring abuts a radial end surface of the central lumen of the introducer locking hub.

Example 62: The introducer sheath system according to any example herein, particularly examples 59-62, wherein the slider includes a guide disposed on an outer surface of the slider body, wherein the guide is movable within a corresponding guide channel provided on the central lumen of the introducer locking hub, the guide channel extends in a longitudinal direction coplanar with a longitudinal axis of the introducer locking hub such that rotational movement between the slider and the introducer locking hub is prevented, wherein movement of the guide within the guide channel results in a corresponding movement of the distal piece of the introducer between a first position, where the distal piece is adjacent the proximal piece, and a second position, where the distal piece is spaced apart from the proximal piece.

Example 63: The introducer sheath system according to any example herein, particularly example 62, wherein a proximal end of the guide channel includes a locking portion for fixing the axial location of the slider/hypotube with respect to the introducer locking hub/introducer.

Example 64: The introducer sheath system according to any example herein, particularly example 63, wherein, when the guide engages the locking portion, the hypotube and the distal portion of the introducer are maintained in the first position where the distal piece of the introducer is adjacent the proximal piece of the introducer, wherein locking portion of the guide channel extends circumferentially around the central lumen of the introducer locking hub from the proximal end of the guide channel such that rotational movement of the hypotube, and the slider fixedly coupled thereto, causes the guide to move from the guide channel into the locking portion.

Example 65: The introducer sheath system according to any example herein, particularly example 64, wherein the locking portion includes a catch, securing the guide within the locking portion of the guide channel.

Example 66: The introducer sheath system according to any example herein, particularly example 65, wherein the catch extends from a side wall of the locking portion towards the center of the locking channel.

Example 67: The introducer sheath system according to any example herein, particularly examples 16-66, further including a safety lock for securing the distal piece of the introducer in the first position and/or second position.

Example 68: The introducer sheath system according to any example herein, particularly example 67, wherein the safety lock fixes the axial position of the hypotube with respect to the sheath hub.

Example 69: The introducer sheath system according to any example herein, particularly examples 67-68, wherein the safety lock is removably coupled to the hypotube and at least one of the introducer locking hub and the sheath hub.

Example 70: The introducer sheath system according to any example herein, particularly examples 67-69 wherein the safety lock includes an elongated member with a proximal end, a distal end, and a central cavity defined therebetween, the safety lock including a side opening in a first side of the elongated member for receiving at least a portion of the introducer locking hub and hypotube therethrough and into the central cavity, wherein the proximal end of the safety lock includes an opening for receiving the hypotube therethrough and retaining the proximal end of the hypotube, wherein the distal end of the safety lock includes an opening for receiving at least one of the introducer locking hub and the sheath hub.

Example 71: A method of delivering a prosthetic device to a procedure site, the method comprising: providing an introducer locking hub having an elongated introducer coupled to a hub body of the locking hub, the introducer comprising a proximal piece and a separate distal piece, the proximal piece being coupled to the hub body of the locking hub, the separate distal piece being coupled to a hypotube, the hypotube slidably disposed within a central lumen of the proximal piece; advancing the introducer through the central lumen of an expandable sheath, the expandable sheath defining an elongated tubular body coupled to a sheath hub and a distal tip portion provided at a distal end of the elongated tubular body; coupling the introducer locking hub to a sheath hub such that the axial position of the introducer locking hub and the sheath hub is fixed; inserting the coupled sheath and introducer at least partially into the vasculature of the patient; advancing the hypotube within the central lumen of the proximal piece of the introducer such that the distal piece moves from a first position, adjacent the proximal piece, to a second position where the distal piece is spaced from the proximal piece; expanding the distal tip portion of the expandable sheath; withdrawing the hypotube within the central lumen of the proximal piece of the introducer such that the distal piece of the introducer abuts the proximal piece of the introducer; withdrawing the introducer from the central lumen of the sheath; advancing a medical device through the central lumen of the sheath; and delivering the medical device to a procedure site via the central lumen of the sheath.

Example 72: The method according to any example herein, particularly example 71, wherein coupling the introducer locking hub to the sheath hub further includes: advancing the hypotube within the central lumen of the expandable sheath such that the distal piece of the introducer extends through and beyond the distal end of the expandable sheath; compressing the distal tip portion of the expandable sheath against a distal end surface of the proximal piece of the introducer; withdrawing the hypotube within the central lumen of the proximal piece of the introducer such that the distal tip portion is distal tip portion is retained between the proximal piece and the distal piece of the introducer.

Example 73: The method according to any example herein, particularly examples 71-72, wherein the hypotube includes a locking mechanism for fixing the introducer in at least one of the first and second positions, wherein the locking mechanism includes a slider fixedly coupled to the hypotube, the slider comprising a slider body having a proximal end and a distal end and defining a central lumen extending longitudinally between the proximal and distal end and a guide disposed on an outer surface of the slider body, wherein the guide is movable within a corresponding guide channel provided on the central lumen of the introducer locking hub, the guide channel extending helically around the central lumen.

Example 74: The method according to any example herein, particularly example 73, wherein the slider further includes a restrictor projection disposed on the outer surface of the slider body, wherein the restrictor projection is axially translatable within a corresponding restrictor channel that extends longitudinally along an inner surface of the sheath hub such that rotational movement between the hypotube (and slider) is prevented, wherein advancing the hypotube within the central lumen of the expandable sheath includes: moving the hypotube in a distal direction such that the restrictor projection moves a corresponding length within the restrictor channel.

Example 75: The method according to any example herein, particularly example 74, wherein withdrawing the hypotube within the central lumen of the expandable sheath includes: moving the hypotube in a proximal direction such that the restrictor projection moves a corresponding length within the restrictor channel.

Example 76: The method according to any example herein, particularly examples 71-75, wherein advancing the hypotube within the central lumen of the expandable sheath includes: rotating introducer locking hub with respect to the hypotube (and/or slider) in a first direction to result in a corresponding axial movement of the guide along the guide channel, where movement of the guide within the guide channel results in a corresponding movement of the distal piece of the introducer from the first position, where the distal piece is adjacent the proximal piece, to the second position, where the distal piece is spaced apart from the proximal piece.

Example 77: The method according to any example herein, particularly examples 71-75, wherein withdrawing the hypotube within the central lumen of the sheath includes: rotating introducer locking hub with respect to the hypotube (and/or slider) in a second direction to result in a corresponding axial movement of the guide along the guide channel, where movement of the guide within the guide channel results in a corresponding movement of the distal piece of the introducer from the second position, where the distal piece is adjacent the proximal piece, to the first position, where the distal piece is spaced apart from the proximal piece.

Example 78: The method according to any example herein, particularly examples 71-77, wherein the hypotube includes a locking mechanism for fixing the introducer in at least one of the first and second positions, wherein the locking mechanism includes a spring disposed around the hypotube between a stop at the proximal end of the hypotube and the introducer locking hub, where the spring biases the hypotube in a proximal direction and the distal piece of the introducer in the second position, wherein advancing the hypotube within the central lumen of the expandable sheath includes: applying a force to the hypotube in a distal direction to overcome the spring bias of the spring, wherein withdrawing the hypotube within the central lumen of the sheath includes: releasing the distal directed force from the hypotube and moving the hypotube in a proximal direction in conjunction with the biasing effect of the spring.

Example 79: The method according to any example herein, particularly example 78, wherein the locking mechanism includes a slider fixedly coupled to the hypotube, the slider comprising a slider body having a proximal end and a distal end and defining a central lumen extending longitudinally between the proximal and distal end and a guide disposed on an outer surface of the slider body, wherein the guide is movable within a corresponding guide channel provided on the central lumen of the introducer locking hub, the guide channel extending in a longitudinal direction coplanar with a longitudinal axis of the introducer locking hub such that rotational movement between the slider and the introducer locking hub is prevented, wherein advancing the hypotube within the central lumen of the expandable sheath further includes: moving the hypotube in a distal direction such that the guide advances a corresponding length within the guide channel.

Example 80: The method according to any example herein, particularly examples 76-79, wherein withdrawing the hypotube within the central lumen of the sheath includes: moving hypotube in a proximal direction such that the guide moves a corresponding length within the guide channel.

Example 81: The method according to any example herein, particularly examples 76-80, wherein a proximal end of the guide channel includes a locking portion for fixing the axial location of the slider/hypotube with respect to the introducer locking hub/introducer, the locking portion extending circumferentially around the central lumen of the introducer locking hub from the proximal end of the guide channel such that rotational movement of the hypotube, and the slider fixedly coupled thereto, causes the guide to move from the guide channel into the locking portion, wherein, when the guide engages the locking portion, the hypotube and the distal portion of the introducer are maintained in the first position where the distal piece of the introducer is adjacent the proximal piece of the introducer, wherein advancing the hypotube within the central lumen of the expandable sheath further includes: rotating the introducer locking hub with respect to the hypotube (and/or slider) in a first direction to move the guide within the locking portion of the guide channel and into the guide channel, moving the hypotube in a distal direction such that the guide advances corresponding length within the guide channel.

Example 82: The method according to any example herein, particularly examples 76-81, wherein withdrawing the hypotube within the central lumen of the sheath includes: moving the hypotube in a proximal direction such that the guide advances corresponding length within the guide channel, rotating the introducer locking hub with respect to the hypotube (and/or slider) in a second direction to move the guide from the guide channel and into the locking portion of the guide channel and into the guide channel.

Example 83: A method of expanding the distal tip of an expandable sheath, the method comprising: providing an introducer locking hub having an elongated introducer coupled to a hub body of the locking hub, the introducer comprising a proximal piece and a separate distal piece, the proximal piece being coupled to the hub body of the locking hub, the separate distal piece being coupled to a hypotube, the hypotube slidably disposed within a central lumen of the hub body; advancing the introducer through the central lumen of an expandable sheath, the expandable sheath defining an elongated tubular body coupled to a sheath hub and a distal tip portion provided at a distal end of the elongated tubular body; coupling the introducer locking hub to a sheath hub such that the axial position of the introducer locking hub and the sheath hub is fixed; advancing the hypotube within the central lumen of the proximal piece of the introducer such that the distal piece moves from a first position, adjacent the proximal piece, to a second position where the distal piece is spaced from the proximal piece; and expanding the distal tip portion of the expandable sheath.

Example 84: The method according to any example herein, particularly examples 83, further including: withdrawing the hypotube within the central lumen of the proximal piece of the introducer such that the distal piece of the introducer abuts the proximal piece of the introducer; and withdrawing the introducer from the central lumen of the sheath.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

What is claimed is:

1. An introducer sheath system comprising:
an introducer locking hub comprising a hub body having a proximal end and a distal end and defining a central lumen extending longitudinally between the proximal end and the distal end;
an introducer coupled to the introducer locking hub, the introducer extending beyond the distal end of the hub body and having a central lumen extending therethrough, the introducer defining an elongated body comprising a proximal piece and a separate distal piece, the proximal piece coupled to the introducer locking hub; and
a hypotube disposed within the central lumen of the introducer, the hypotube having a proximal end and a distal end and defining a central lumen extending longitudinally between the proximal end and the distal end,
wherein the distal end of the hypotube is coupled to the distal piece of the introducer and at least a portion of the hypotube is slidably disposed in the central lumen of the proximal piece of the introducer such that movement of the hypotube within the central lumen of the proximal piece causes the distal piece of the introducer to move with respect to the proximal piece of the introducer.

2. The system of claim 1, wherein the distal piece of the introducer is movable from a first position adjacent the proximal piece to a second position spaced from the proximal piece.

3. The system of claim 2, wherein axial movement of the hypotube within the central lumen of the proximal piece of the introducer causes a corresponding axial movement of the distal piece of the introducer,
wherein, when the introducer is in the first position, axial movement of the hypotube in a distal direction causes the distal piece to move from the first position to the second position,
wherein, when the introducer is in the second position, axial movement of the hypotube in a proximal direction causes the distal piece to move from the second position to the first position.

4. The system of claim 1, wherein rotational movement of the hypotube within the central lumen of the proximal piece of the introducer causes a corresponding rotational movement of the distal piece of the introducer.

5. The system of claim 2, wherein the proximal piece of the introducer includes a tapered distal end, where at least a portion of the tapered distal end of the proximal piece is received within the central lumen of the distal piece when the introducer is in the first position,
wherein the central lumen of the distal piece includes a corresponding tapered surface.

6. The system of claim 1, further comprising an expandable sheath coupled to a sheath hub, the sheath defining an elongated tubular body with a central lumen extending therethrough, the central lumen of the sheath aligned with the central lumens of the sheath hub and the introducer locking hub, wherein the introducer locking hub is removably coupled to the sheath hub and at least a portion of the introducer is disposed within the central lumen of the sheath.

7. The system of claim 6, wherein the sheath includes an expandable body portion and a distal tip portion provided at a distal end of the body portion,
wherein the distal tip portion is biased in an expanded configuration.

8. The system of claim 7, wherein, when the distal piece of the introducer is in a first position adjacent the proximal piece, the distal tip portion of the sheath is retained between the proximal and distal pieces of the introducer,
wherein, when the distal piece of the introducer is in a second position spaced from the proximal piece, the distal tip portion of the sheath is released from the introducer and transitions to the expanded configuration.

9. The system of claim 6, wherein the sheath hub includes a sheath locking sleeve for removably coupling the sheath hub to the introducer locking hub, the sheath locking sleeve including a sleeve body having a proximal end and a distal end and defining a central lumen extending longitudinally between the proximal end and the distal end, a guide disposed on an outer surface of the sleeve body,
wherein the hub body of the introducer locking hub further defines a locking channel disposed on the hub body,
wherein the guide is movable within the locking channel between an unlocked position where the sheath locking sleeve is rotationally and axially movable with respect to the introducer locking hub, and a locked position where the sheath locking sleeve is axially fixed with respect to the introducer locking hub.

10. The system of claim 1, wherein the central lumen of the distal piece includes a large diameter portion adjacent a proximal end of the distal piece, a small diameter portion adjacent a distal end of the distal piece, and a decreasing tapered surface extending between the proximal end of the distal piece and the large diameter portion,
wherein the hypotube is fixedly coupled within the large diameter portion of the central lumen of the distal piece.

11. The system of claim 2, including a locking mechanism for fixing the introducer in at least one of the first and second positions, the locking mechanism including a slider fixedly coupled to the hypotube, the slider comprising a slider body having a proximal end and a distal end and defining a central lumen extending longitudinally between the proximal and distal end, where the slider movably couples the hypotube to the introducer locking hub.

12. The system of claim 11, wherein the slider includes a guide disposed on an outer surface of the slider body,
wherein the guide is movable within a corresponding guide channel provided on the central lumen of the introducer locking hub,
wherein movement of the guide within the guide channel results in a corresponding movement of the distal piece of the introducer between a first position, where the distal piece is adjacent the proximal piece, and a second position, where the distal piece is spaced apart from the proximal piece.

13. The system of claim 11, wherein the slider includes a second guide disposed on an outer surface of the slider body, the second guide circumferentially opposite the guide,
wherein the second guide is movable within a corresponding second guide channel provided on the central lumen of the introducer locking hub,
wherein movement of the second guide within the second guide channel results in a corresponding movement of the distal piece of the introducer between the first position and the second position.

14. The system of claim 11, further including an expandable sheath coupled to a sheath hub, the sheath defining an elongated tubular body with a central lumen extending therethrough, the central lumen of the sheath aligned with the central lumens of the sheath hub and the introducer locking hub,
wherein the slider includes a restrictor projection disposed on an outer surface of the slider body,
wherein the restrictor projection is movable within a corresponding restrictor channel provided on the central lumen of the sheath hub.

15. The system of claim 14, wherein the sheath hub includes a locking sleeve for removably coupling the sheath hub to the introducer locking hub,
wherein the sheath locking sleeve comprises a sleeve body having a proximal end and a distal end and defining a central lumen extending longitudinally between the proximal end and the distal end, a guide disposed on an outer surface of the sleeve body,
wherein the restrictor channel is provided on the central lumen of the locking sleeve.

16. The system of claim 15, wherein, when the sheath hub is coupled to the introducer locking hub, axial movement between the expandable sheath and the introducer locking hub is prevented,
wherein the restrictor channel extends in a longitudinal direction coplanar with a longitudinal axis of the sheath hub such that rotational movement between the slider and the sheath hub is prevented,
wherein rotational movement of the introducer locking hub with respect to the slider results in a corresponding axial movement of the guide along the guide channel, where movement of the guide within the guide channel results in a corresponding movement of the distal piece of the introducer between a first position, where the distal piece is adjacent the proximal piece, and a second position, where the distal piece is spaced apart from the proximal piece.

17. The system of claim 11, wherein the locking mechanism includes a spring disposed around the hypotube between a stop at the proximal end of the hypotube and the introducer locking hub,
wherein the spring biases the hypotube in a proximal direction and the distal piece of the introducer in the first position.

18. A method of delivering a prosthetic device to a procedure site, the method comprising:
providing an introducer locking hub having an elongated introducer coupled to a hub body of the locking hub, the introducer comprising a proximal piece and a separate distal piece, the proximal piece being coupled to the hub body of the locking hub, the separate distal piece being coupled to a hypotube, the hypotube slidably disposed within a central lumen of the proximal piece;
advancing the introducer through the central lumen of an expandable sheath, the expandable sheath defining an elongated tubular body coupled to a sheath hub and a distal tip portion provided at a distal end of the elongated tubular body;
coupling the introducer locking hub to a sheath hub such that an axial position of the introducer locking hub and the sheath hub is fixed;
inserting the coupled sheath and introducer at least partially into a vasculature of a patient;
advancing the hypotube within the central lumen of the proximal piece of the introducer such that the distal piece moves from a first position, adjacent the proximal piece, to a second position where the distal piece is spaced from the proximal piece;
expanding the distal tip portion of the expandable sheath;
withdrawing the hypotube within the central lumen of the proximal piece of the introducer such that the distal piece of the introducer abuts the proximal piece of the introducer;
withdrawing the introducer from the central lumen of the sheath;
advancing a medical device through the central lumen of the sheath; and
delivering the medical device to a procedure site via the central lumen of the sheath.

19. The method of claim 18, wherein coupling the introducer locking hub to the sheath hub further includes:
advancing the hypotube within the central lumen of the expandable sheath such that the distal piece of the introducer extends through and beyond the distal end of the expandable sheath;
compressing the distal tip portion of the expandable sheath against a distal end surface of the proximal piece of the introducer;
withdrawing the hypotube within the central lumen of the proximal piece of the introducer such that the distal tip portion is distal tip portion is retained between the proximal piece and the distal piece of the introducer.

20. A method of expanding a distal tip of an expandable sheath, the method comprising:
providing an introducer locking hub having an elongated introducer coupled to a hub body of the locking hub, the introducer comprising a proximal piece and a separate distal piece, the proximal piece being coupled to the hub body of the locking hub, the separate distal piece being coupled to a hypotube, the hypotube slidably disposed within a central lumen of the hub body;
advancing the introducer through the central lumen of an expandable sheath, the expandable sheath defining an elongated tubular body coupled to a sheath hub and a distal tip portion provided at a distal end of the elongated tubular body;
coupling the introducer locking hub to a sheath hub such that an axial position of the introducer locking hub and the sheath hub is fixed;
advancing the hypotube within the central lumen of the proximal piece of the introducer such that the distal piece moves from a first position, adjacent the proximal piece, to a second position where the distal piece is spaced from the proximal piece; and
expanding the distal tip portion of the expandable sheath.
